SEQUENCE LISTING (2 sequences)

SEQ ID NO: 1 - DNA, 2446 bp, Rattus sp.
  exon (57)..(2343)
gccaagcgcgcaattaaccctcactaaagggaacaaaagctgggtaccttgtcgacatgaagttgtggctgaactggattttccttgtccttttcataaaaggtgtcaagtgtgaggtgtacctggtagagtctgggggcggtttagtgcagcctggcgggtccgtgaaactctcctgttcagcctcaggattcactttcagtacctttccaatggcctgggtccgccaggctccaacgcagggtctgcagtgggtcgcaacccttagtcctagtggtgatagcacttactatcgagattccgtgaagggccgattcactatctccagagataatgtactaaacaccctatacctccacatggacattctgaggtctgaggacacggccacttattactgtacaaaagtcagatttaccaccttctattttgattcctggggccaaggagtcatggtcgcagtctcctcagctgaaacaacagccccatctgtctatccactggctcctggaactgctctcaaaagtaactccatggtgaccctgggatgcctggtcaagggctatttccctgagccagtcaccgtgacctggaactctggagccctgtccagcggtgtgcacaccttcccagctgtcctgcagtctggactctacactctcaccagctcagtgactgtaccctccagcacctggtccagccaggccgtcacctgcaacgtagcccacccggccagcagcaccaaggtggacaagaaaatttcctcggcggatgatgctaagaaggatgctgctaagaaggatgatgctaagaaagatgatgctaagaaagatgcaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaaactgagcaaagaccccaacgagaagcgcgatcacatggtcctgaaagagttcgtgaccgccgccgggatcactctcggcatggacgagctgtacaagccgcgggccaagcgagcaccggtgaaacagactttgaattttgaccttctcaagttggcaggagacgttgagtccaaccctgggcccatgatggctccagttcaactcttagggctgctgctgctctggctcccagccatgagatgtgacatccagatgacccagtctccttcattcctgtctgcatctgtgggagacagagtaactatcaactgcaaagcaagtcagaatattaacaagtacttggactggtatcagcaaaagtttggtgagactcccaaactcctgatatataatataaacaatttgcattcaggagtcccatcaaggttcagtggcagtggatctggtcctgatttcacacttaccatcagcagcctgcagcctgaagatgttgccacatatttctgccttcaacatcgcactgggtggacgttcggtggaggcaccaaggtggaattgagacgggctgatgctgcaccaactgtatccatcttcccaccatccatggaacagttaacatctggaggtgccacagtcgtgtgcttcgtgaacaacttctatcccagagacatcagtgtcaagtggaagattgatggcagtgaacaacgagatggtgtcctggacagtgttactgatcaggacagcaaagacagcacgtacagcatgagcagcaccctctcgttgaccaaggttgaatatgaaaggcataacctctatacctgtgaggttgttcataagacatcatcctcacccgtcgtcaagagcttcaacaggaatgagtgttaggtaagtacatctagagagctccaattcgccctatagtgagtcgtattacgcgcgctcactggccgtcgttttacaacgtcgtgactgggaaaaccctggcgtta

SEQ ID NO: 2 - DNA, 2445 bp, Rattus sp.
cggttcgcgcgttaattgggagtgatttcccttgttttcgacccatggacagctgtacttcaacaccgacttgacctaaaaggaacaggaaaagtattttccacagttcacactccacatggaccatctcagacccccgccaaatcacgtcggaccgcccaggcactttgagaggacaagtcggagtcctaagtgaaagtcatggaaaggttaccggacccaggcggtccgaggttgcgtcccagacgtcacccagcgttgggaatcaggatcaccactatcgtgaatgatagctctaaggcacttcccggctaagtgatagaggtctctattacatgatttgtgggatatggaggtgtacctgtaagactccagactcctgtgccggtgaataatgacatgttttcagtctaaatggtggaagataaaactaaggaccccggttcctcagtaccagcgtcagaggagtcgactttgttgtcggggtagacagataggtgaccgaggaccttgacgagagttttcattgaggtaccactgggaccctacggaccagttcccgataaagggactcggtcagtggcactggaccttgagacctcgggacaggtcgccacacgtgtggaagggtcgacaggacgtcagacctgagatgtgagagtggtcgagtcactgacatgggaggtcgtggaccaggtcggtccggcagtggacgttgcatcgggtgggccggtcgtcgtggttccacctgttcttttaaaggagccgcctactacgattcttcctacgacgattcttcctactacgattctttctactacgattctttctacgttaccactcgttcccgctcctcgacaagtggccccaccacgggtaggaccagctcgacctgccgctgcatttgccggtgttcaagtcgcacaggccgctcccgctcccgctacggtggatgccgttcgactgggacttcaagtagacgtggtggccgttcgacgggcacgggaccgggtgggagcactggtgggactggatgccgcacgtcacgaagtcggcgatggggctggtgtacttcgtcgtgctgaagaagttcaggcggtacgggcttccgatgcaggtcctcgcgtggtagaagaagttcctgctgccgttgatgttctgggcgcggctccacttcaagctcccgctgtgggaccacttggcgtagctcgacttcccgtagctgaagttcctcctgccgttgtaggaccccgtgttcgacctcatgttgatgttgtcggtgttgcagatatagtaccggctgttcgtcttcttgccgtagttccacttgaagttctaggcggtgttgtagctcctgccgtcgcacgtcgagcggctggtgatggtcgtcttgtgggggtagccgctgccggggcacgacgacgggctgttggtgatggactcgtgggtcaggtttgactcgtttctggggttgctcttcgcgctagtgtaccaggactttctcaagcactggcggcggccctagtgagagccgtacctgctcgacatgttcggcgcccggttcgctcgtggccactttgtctgaaacttaaaactggaagagttcaaccgtcctctgcaactcaggttgggacccgggtactaccgaggtcaagttgagaatcccgacgacgacgagaccgagggtcggtactctacactgtaggtctactgggtcagaggaagtaaggacagacgtagacaccctctgtctcattgatagttgacgtttcgttcagtcttataattgttcatgaacctgaccatagtcgttttcaaaccactctgagggtttgaggactatatattatatttgttaaacgtaagtcctcagggtagttccaagtcaccgtcacctagaccaggactaaagtgtgaatggtagtcgtcggacgtcggacttctacaacggtgtataaagacggaagttgtagcgtgacccacctgcaagccacctccgtggttccaccttaactctgcccgactacgacgtggttgacataggtagaagggtggtaggtaccttgtcaattgtagacctccacggtgtcagcacacgaagcacttgttgaagatagggtctctgtagtcacagttcaccttctaactaccgtcacttgttgctctaccacaggacctgtcacaatgactagtcctgtcgtttctgtcgtgcatgtcgtactcgtcgtgggagagcaactggttccaacttatactttccgtattggagatatggacactccaacaagtattctgtagtaggagtgggcagcagttctcgaagttgtccttactcacaatccattcatgtagatctctcgaggttaagcgggatatcactcagcataatgcgcgcgagtgaccggcagcaaaatgttgcagcactgacccttttgggaccgcaat

(19) United States
(12) Patent Application Publication
(10) Pub. No.: US 2011/0185441 A1
(43) Pub. Date: Jul. 28, 2011

(54) TRANSGENIC ANIMALS AND METHODS OF MAKING RECOMBINANT ANTIBODIES

(75) Inventors: François Romagne, La Ciotat (FR); Bernard Malissen, Marseille (FR)

(73) Assignees: INNATE PHARMA, S.A., MARSEILLE (FR); INSERM (INSTITUT NATIONAL DE LA SANTE ET DE LA RECHERCHE MEDICALE), PARIS CEDEX (FR)

(21) Appl. No.: 13/082,520

(22) Filed: Apr. 8, 2011

Related U.S. Application Data
(63) Continuation of application No. 11/912,553, filed on Oct. 25, 2007.
(60) Provisional application No. 60/676,574, filed on Apr. 29, 2005.
(60) Provisional application No. 60/730,350, filed on Oct. 26, 2005.

(51) Int. Cl. A01K 67/027

(52) U.S. Cl. 800/18

(57) ABSTRACT

The present invention describes a means for obtaining cells which produce human, humanized or chimeric antibodies in commercially useful quantities. The invention permits high antibody producer cells to be selected and isolated from animals for use in culture to produce antibodies. The invention also provides methods for the affinity maturation of human, humanized or chimeric immunoglobulins.

### CROSS-REFERENCE TO RELATED APPLICATIONS

This application is a continuation of U.S. application Ser. No. 11/912,553, filed Oct. 25, 2007, which is the U.S. national stage application of International Patent Application No. PCT/IB2006/001912, filed Apr. 28, 2006, which claims the benefit of U.S. Provisional Patent Application Nos. 60/676,574, filed Apr. 29, 2005 and 60/730,350, filed Oct. 26, 2005 the disclosures of which are hereby incorporated by reference in their entirety, including all figures, tables and amino acid or nucleic acid sequences.

The Sequence Listing for this application is labeled “seq-list.txt”, which was created on Sep. 22, 2010, and is 15 KB. The entire contents is incorporated herein by reference in its entirety.

### FIELD OF THE INVENTION

Humanized, human or chimeric immunoglobulins that are reactive with specific antigens are promising therapeutic and/or diagnostic agents. However, producing sufficient quantities of human, humanized and/or chimeric antibodies has proved difficult. The subject application provides a means for the production of human, humanized or chimeric antibodies in commercially useful quantities. The invention permits high antibody producer cells to be selected and isolated from animals for use in culture to produce antibodies. The invention also provides methods for the affinity maturation of human, humanized or chimeric immunoglobulins.

### BACKGROUND

The basic immunoglobulin (Ig) structural unit in vertebrate systems is composed of two identical “light” polypeptide chains (approximately 23 kDa), and two identical “heavy” chains (approximately 53 to 70 kDa). Heavy and light chains are joined by disulfide bonds in a “Y” configuration, and the “tail” portions of the two heavy chains are also bound by covalent disulfide linkages.

Light and heavy Ig chains are each composed of a variable region at the N-terminal end, and a constant region at the C-terminal end. In the light chain, the variable region (termed “V_{L} J_{L}”) is composed of a variable (V_{L}) region connected through the joining (J_{L}) region to the constant region (C_{L}). In the heavy chain, the variable region (V_{H} D_{H} J_{H}) is composed of a variable (V_{H}) region linked through a combination of the diversity (D_{H}) region and the joining (J_{H}) region to the constant region (C_{H}). The V_{L} J_{L} and V_{H} D_{H} J_{H} regions of the light and heavy chains, respectively, are associated at the tips of the Y to form the antibody's antigen binding portion and determine antigen binding specificity.

The (C_{H}) region defines the antibody's isotype, i.e., its class or subclass. Antibodies of different isotypes differ significantly in their effector functions, such as the ability to activate complement, bind to specific receptors (e.g., Fc receptors) present on a wide variety of cell types, cross mucosal and placental barriers, and form polymers of the basic four-chain IgG molecule.

Antibodies are categorized into “classes” according to the C_{H} type utilized in the immunoglobulin molecule (IgM, IgG, IgD, IgE, or IgA). There are at least five types of C_{H}. genes (Cμ, Cγ, Cδ, Cε, and Cα), and some species (including humans) have multiple C_{H} subtypes (e.g., Cγ₁, Cγ₂, Cγ₃, and Cγ₄ in humans for IgG subtypes). There are a total of nine C_{H} genes in the haploid genome of humans, eight in mouse and rat, and several but fewer in many other species. In contrast, there are normally only two types of light chain constant regions (C_{L}), kappa (κ) and lambda (λ), and only one of these constant regions is present in a single light chain protein (i.e., there is only one possible light chain constant region for every V_{L} J_{L} produced). Each heavy chain class can be associated with either of the light chain classes (e.g., a C_{H} γ region can be associated with either a kappa. or lambda. light chain in a given antibody). The constant regions of the heavy and light chains within a particular class do not participate to antigen binding site and therefore to antigen specificity.

Each of the V, D, J, and C regions of the heavy and light chains are encoded by distinct genomic sequences or gene segments. Antibody diversity is generated by recombination between the different V_{H}, D_{H}, and J_{H} gene segments in the heavy chain, and V_{L} and J_{L} gene segments in the light chain. The recombination of the different V_{H}, D_{H}, and J_{H} genes is accomplished by DNA recombination during B cell differentiation. Briefly, the heavy chain sequence recombines first to generate a D_{H} J_{H} complex, and then a second recombinatorial event produces a V_{H} D_{H} J_{H} complex. A functional heavy chain is produced upon transcription followed by splicing of the RNA transcript. Production of a functional heavy chain triggers recombination in the light chain sequences to produce a rearranged V_{L} J_{L} region which in turn forms a functional V_{L} J_{L} C_{L} region, i.e., the functional light chain. Besides recombination, two additional phenomenon increase the diversity and are known in the art as N diversity (trimming and addition of nucleotides at the V/D/J junctions) and somatic hypermutation (high degree of additional mutations in the rearranged VDJ segment when a mature B cell encounters an antigen, that results in increasing the affinity of the mutated IgG towards this antigen).

During the course of B cell differentiation, progeny of a single B cell can switch the expressed immunoglobulin isotype from IgM to IgG or other classes of immunoglobulin without changing the antigen specificity determined by the variable region. This phenomenon, known as immunoglobulin class-switching, is accompanied by DNA rearrangement that takes place between switch (S) regions located 5′ to each C_{H} gene (except for Cγ) (reviewed in Honjo (1983) Annu. Rev. Immunol. 1:499-528, and Shimizu & Honjo (1984) Cell 36:801-803). S-S recombination brings the V_{H} D_{H} J_{H} exon to the proximity of the C_{H} gene to be expressed by deletion of intervening C_{H} genes located on the same chromosome. The class-switching mechanism is directed by cytokines (Mills et al. (1995) J. Immunol. 155:3021-3036). Switch regions vary in size from 1 kb (SE) to 10 kb (Sγ₁), and are composed of tandem repeats that vary both in length and sequence (Gritzmacher (1989) Crit. Rev. Immunol. 9:173-200). Several switch regions have been characterized including the murine Sμ, Sε, Sγ, Sγ3, Sγ1, Sγ2b and Sγ2a switch regions and the human Sμ switch region (Mills et al. (1995) Supra; Nikaido et al. (1981) Nature 292:845-8; Marcu et al. (1982) Nature 298:87-89; Takahashi et al. (1982) Cell 29:671-9; Mills et al. (1990) Nucleic Acids Res. 18:7305-16; Nikaido et al. (1982) J. Biol. Chem. 257:7322-29; Stanton et al. (1982) Nucleic Acids Res. 10:5993-6006; Gritzmacher (1989) supra; Davis et al. (1980) Science 209:1360; Obata et al. (1981) Proc. Natl. Acad. Sci. U.S.A. 78:2437-41; Kataoka et al. (1981) Cell 23:357; Mowatt et al. (1986) J. Immunol. 136:2674-83; Szurek et al. (1985) J. Immunol. 135:620146; and Wu et al. (1984) EMBO J. 3:2033-40).

The value and potential of antibodies as diagnostic and therapeutic reagents has been long- recognized in the art. Unfortunately, the field has been hampered by the slow, tedious processes required to produce large quantities of an antibody of a desired specificity. The classical cell fusion techniques allowed for efficient production of monoclonal antibodies by fusing the B cell producing the antibody with an immortalized cell line. The resulting cell line is called a hybridoma cell line. However, most of these monoclonal antibodies are produced in murine systems and are recognized as “foreign” proteins by the human immune system. Thus the patient's immune system elicits a response against the antibodies, which results in antibody neutralization and clearance, and/or potentially serious side-effects associated with the anti-antibody immune response.

One approach to this problem has been to develop chimeric, human and “humanized” monoclonal antibodies, which are not as easily “recognized” as foreign epitopes, and avoid an anti-antibody immune response in the patient. However, the technologies for production of human or humanized antibodies each face certain constraints and disadvantages. Chimeric, human and humanized antibodies must be expressed in recombinant production systems (e.g. CHO cell systems), necessitating a development of cell lines capable of producing sufficient amounts of antibody under conditions that can be used in large scale production; these production systems also involve extensive characterization for regulatory purposes. Examples of techniques which rely upon recombinant DNA techniques such as those described above to produce chimeric antibodies are described in PCT Publication No. WO 86/01533 (Neuberger et al.), and in U.S. Pat. No. 4,816,567 (Cabilly et al.) and U.S. Pat. No. 5,202,238 (Fell et al.). These methods require transferring DNA from one cell to another, thus removing it from its natural locus, and thus require careful in vitro manipulation of the DNA to ensure that the final antibody-encoding construct is functional (e.g., is capable of transcription and translation of the desired gene product). Failure to produce amounts of antibody compatible with clinical practice in those transfectants is a common reason for failure of antibody based programs. In comparison, B cell hybridoma-based production has been well characterized and usually provides high amount of monoclonal antibody, and thus would offer a more straightforward production process. There is a clear need in the field for a method for producing a desired protein or antibody which does not require multiple cloning steps, in more efficient systems than conventional transfection systems, and can be carried out from hybridoma cells.

Another often used technology is based on transgenic mice carrying a human Ig locus. These mice produce human antibody producing B cells; although in some cases the B cell can be fused to generate a hybridoma, most B cells obtained are not suitable for production and recombinatory techniques as described above must be employed. Moreover, the transgenic mouse system does not allow an antibody against a target antigen to be obtained and does not permit development based on a lead antibody (e;g; a known human, chimeric or rodent mAb with interesting properties). For example, many human tumor antigens are not immunogenic in mice and it is therefore difficult to isolate B cells producing antibodies against human antigens from these animals. Finally, even in those instances where it is possible to obtain B cells from such transgenic animals that can be fused to produce a hybridoma that can be used in production, the B cells generally provide low levels of antibody production.

Additionally, beyond the basic problem of expression of antibodies (e.g. obtaining high-producing B cells or having to use non-hybridoma cells such as CHO cells in production), many antibodies obtained using classical immunization procedures lack affinity or other characteristics desired in an antibody intended for therapeutic use. For example, in some cases upon immunization, IgM but not IgG antibody producing B cells are obtained (IgM antibodies generally having low affinity). In other cases, IgG producing B cells are obtained but the antibodies lack the desired affinity. In yet other cases, the humanization or chimerisation (e.g. CDR-grafting) process results in decreased affinity. One well known approach is phage display technology, used to generate large libraries of antibody fragments by exploiting the capability of bacteriophage to express and display biologically functional protein molecule on its surface. Combinatorial libraries of antibodies have been generated in bacteriophage lambda expression systems which may be screened as bacteriophage plaques or as colonies of lysogens (Huse et al. (1989) Science 246: 1275; Caton and Koprowski (1990) Proc. Natl. Acad. Sci. (U.S.A.) 87: 6450). Various embodiments of bacteriophage antibody display libraries and lambda phage expression libraries have been described (Kang et al. (1991) Proc. Natl. Acad. Sci. (U.S.A.) 88: 4363; Clackson et al. (1991) Nature 352: 624; McCafferty et al. (1990) Nature 348: 552 Generally, a phage library is created by inserting a library of random oligonucleotides or a cDNA library encoding antibody fragment such as VL and VH into gene 3 of M13 or fd phage. Each inserted gene is expressed at the N-terminal of the gene 3 product, a minor coat protein of the phage. As a result, peptide libraries that contain diverse peptides can be constructed. The phage library is then affinity screened against immobilized target molecule of interest, such as an antigen, and specifically bound phage particles are recovered and amplified by infection into Escherichia coli host cells. Typically, the target molecule of interest such as a receptor (e.g., polypeptide, carbohydrate, glycoprotein, nucleic acid) is immobilized by a covalent linkage to a chromatography resin to enrich for reactive phage particles by affinity chromatography and/or labeled for screening plaques or colony lifts. This procedure is called biopanning Finally, high affinity phage clones can be amplified and sequenced for deduction of the specific peptide sequences. A number of “affinity maturation” or other solutions have been developed to deal with this problem, but to date all remain tedious and time consuming. There is therefore a need in the art for methods permitting the modification of a candidate antibody in order to improve its antigen binding properties.

### BRIEF SUMMARY OF THE INVENTION

The subject invention provides transgenic animals useful for the production of human, humanized or chimeric antibodies. Transgenic animals provided herein include: 1) “light (L) chain only animals”; 2) “heavy (H) chain only animals”; and 3) “progeny animals” arising from the mating of “light chain only animals” and “heavy chain only animals”. Also provided by the subject invention are human, humanized or chimeric antibodies produced by B-cells of said progeny animals and isolated B-cells producing such antibodies from said progeny animals. The subject invention also provides immortalized cell lines that produce human, humanized or chimeric antibodies of various specificities prepared from B-cells of said progeny animals.

The invention encompasses a light (L) chain only animal comprising a rearranged V-J portion of a selected immunoglobulin light chain placed (introduced) into its germline DNA and a heavy chain (H) only animal comprising a rearranged V_{H}DJ_{H} portion of the selected immunoglobulin (i.e. a human, chimeric, rodent or other species mAb of known specificity) heavy chain placed (introduced) into its germline DNA. Also encompassed are progeny animals arising from the mating of said light chain only animals and heavy chain only animals. Preferably the germline DNA of said progeny animals will comprise a rearranged V-J portion of a selected immunoglobulin light chain and a rearranged V_{H}DJ_{H} portion of the selected immunoglobulin heavy chain.

In another embodiment, the invention provides a heavy (H) chain only animal, preferably a mouse or rat, comprising a rearranged V_{H}DJ_{H} portion of a selected immunoglobulin heavy chain placed (introduced) upstream of a murine μ constant region, and a sequence encoding a human heavy chain constant region replacing the murine germline DNA that encodes one or more of the murine heavy chain constant regions (for example replacing the murine α region, the murine Cγ3, Cγ1, Cγ2b and Cγ2a region set, and/or the ε heavy chain constant region). The human heavy chain constant region sequence is operably linked to a switch sequence. For example, when a human ε or γ heavy chain constant region sequence replaces a murine ε heavy chain constant region, an arrangements as follows can be constructed:
5′-Sε-human Cε-Sα-Cα-3′, or
5′-Sε-human Cγ-Sα-Cα-3′,
   wherein C represents a constant region, γ may be any human γ constant region subtype G1, G2, G3 or G4 or portion thereof, S represents a switch sequence, and Sε, Sα-Cα may be of human or nonhuman (e.g. murine) origin. The invention also provides a light (L) chain only animal comprising a rearranged V-J portion of a selected immunoglobulin light chain placed (introduced) into its germline DNA, preferably light (L) chain only mouse comprising a rearranged V-J portion of a selected immunoglobulin light chain upstream of a murine C_{L}κ or C_{L}λ sequence, preferably with mouse C_{L}κ or C_{L}λ sequences being replaced by human C_{L}κ or C_{L}ζ sequences.

Also encompassed are progeny animals arising from the mating of said light chain only animals and heavy chain only animals.

A number of preferred examples can be envisioned, which are further described herein. The invention provides numerous advantages which include but are not limited to the following. Many of the advantages arise from the possibility, as a result of modifications in the germline DNA of transgenic animals of the invention, to express an antibody of interest (a predetermined antibody) by a non-human B cell from its natural Ig heavy and light chain locus. Firstly, in one configuration the invention provides that progeny animals can be obtained which have a set of B cells that produce only a single species of antibody of interest. This permits the most desirable antibody-producing cells to be selected among a large number of B cells. Production of an antibody of interest (e.g. an antibody for which the sequence of its specificity is known) can then be envisioned from such a high producer cell line, generally after immortalization. Thus, antibodies of interest will preferably be expressed under the control of native (to the species of origin of the cell) regulatory sequences when the animals, vectors and cells of the invention retain the native regulatory control sequences (e.g. mouse, rat). It will be appreciated however that non-native (e.g. human) immunoglobulin regulatory sequences can be used as well. Because B cells when immortalized are well suited for production this permits commercial production cell lines to be obtained. Current methods require either production from cell lines obtained from the initial immunization when the antibody was obtained, or transfection of DNA encoding the heavy and light chains of antibodies into certain production cell lines (e.g. CHO, myeloma). None of these current methods are satisfactory. Moreover, when an antibody is modified, as in the case of chimeric, humanized and CDR grafted antibodies, the constructs necessarily have to be transfected to host cells. Furthermore, in some cases, glycosylation changes may occur when an antibody of interest is expressed in a production host cell. For example, hybridomas obtained from rats have been reported to have different glycosylation from that produced by murine cell lines (e.g. CHO), and for some rat originated antibodies the murine cell lines produced increased fucose content, which in turn is known to result in decreased ADCC (antibody dependent cellular cytotoxicity) activity toward a target cell. Thus for some antibodies where glycoylation differs in a production cell from that in the initial hybridoma, it would be advantageous to produce new cell lines using the present methods, which could be used in commercial production.

The method of the invention furthermore provide for the ability to produce a predetermined antibody from a cell which does not produce other antibodies, as may occur from its endogenous immunoglobulin genes. For a number of antibody types of commercial interest such as humanized, chimeric, or antibodies having a constant region isotype different from that of the lead antibody this is generally not possible to date. It can also be advantageous to generate antibodies with constant chains linked to other proteins, for example fluorescent proteins; a precise ratio of antibody to marker is important in diagnostic and research applications.

The invention also provides other advantages. For example, a single progeny animal can produce different cells that produce antibody of different formats. By creating an animal with a rearranged variable region for the antibody of interest linked to multiple constant regions of interest, the expression of which is under the control of switch regions, it is possible to express an antibody(ies) of interest having any desired isotype, constant regions from other species, or constant regions linked to detectable markers. This is useful in pharmaceutical development, for example, where it is often desirable to generate both a full antibody and an antibody fragment of the same lead antibody in order to distinguish between effects mediated by the constant region (e.g. depleting cells to which the antibody is bound). Uses can also be found in diagnostics and research, where cells can be obtained that produce the same antibody without a detectable marker, and in a format linked to a marker.

In yet further advantages, as a result of the possibility to induce somatic hypermutation of the variable regions in the progeny animals of the invention, the invention also provides for modification and improvement of an antibody of interest. An antibody having for example low affinity for its antigen can be improved by the somatic hypermutation, thus providing an affinity maturation.

The invention also provides a targeting construct comprising a sequence of a rearranged. V_{H}DJ_{H} portion of a selected immunoglobulin heavy chain placed upstream of a murine μ constant region, a sequence encoding a human heavy chain constant region replacing the murine germline DNA that encodes one or more of the murine heavy chain constant regions (for example replacing the murine α region, the set of murine Cγ3, Cγ1, Cγ2b and Cγ2a regions, and/or the murine κ heavy chain constant region) and two homology arms. Said sequence encoding a human heavy chain constant region is preferably operably linked to a switch sequence. Said targeting construct comprises at least a portion of a murine IgH locus into which said rearranged V_{H}DJ_{H} portion and said sequence encoding a human heavy chain constant region have to be inserted. The invention also provides a second targeting construct comprising a rearranged V-J portion of a selected immunoglobulin light chain, upstream of a Ckappa (also referred to as C_{L}κ or Igε) or Clambda (also referred to as C_{L}λ or Igλ) light chain sequence, the C_{L}λ and C_{L}λ sequences preferably being of murine or human origin, and two homology arms. In a particular embodiment, said second targeting construct comprises a rearranged V-J portion of a selected immunoglobulin light chain upstream of a human Ckappa (also referred to as C_{L}κ or Igκ) or Clambda (also referred to as C_{L}λ or Igλ) light chain sequence and two homology arms.

Also provided is therefore a set of targeting constructs comprising said first and second targeting constructs. The first and second targeting constructs will optionally comprise a sequence encoding a selectable marker, and a immunoglobulin (Ig) promoter that can drive expression of the Ig genes included in the targeting constructs. The targeting constructs can also contain the recognition, amplification and/or target sequences already mentioned. Optionally, the targeting construct can also comprise a negative selectable marker outside of the two homology arms.

Another object of the present invention is the stably transfected embryonic stem (ES) cell clone produced by transfecting a cell with said first or said second targeting constructs, as well as a method of creating a transgenic nonhuman mammal with said stably transfected embryonic stem (ES) cell clones. According to the latter method the stably transfected ES cell clones according to the invention are injected into mouse blastocysts, these blastocysts are transferred to the surrogate mother, the animals born therefrom are mated and their offspring selected for the presence of the mutation. These offspring will be either light (L) chain only animals” or “heavy (H) chain only animals” depending on whether they have inserted into their germline DNA the sequences from the first or the second targeting vector. Transgenic nonhuman animals that can be obtained in this fashion are also an object of the present invention.

“Progeny animals” arising from the mating of “light chain only animals” and “heavy chain only animals” are selected for presence of the sequences from both the first and second targeting vector. Transgenic nonhuman animals that can be obtained in this fashion are also an object of the present invention; such animals therefore comprise in their germline DNA (a) a rearranged V_{H}DJ_{H} portion of a selected immunoglobulin heavy chain placed upstream of the murine μ constant region, (b) a sequence encoding a human heavy chain constant region replacing the murine germline DNA that encodes one or more heavy chain constant regions (for example replacing the murine α region, the set of the murine Cγ3, Cγ1, Cγ2b and Cγ2a regions, and/or the ε heavy chain constant region) preferably operably linked to a switch sequence, and (c) a rearranged V-J portion of a selected immunoglobulin light chain, upstream of a murine C_{L}κ or C_{L}λ sequence, preferably with human C_{L}κ or C_{L}λ sequences replacing the murine C_{L}κ or C_{L}λ sequences. Another object of the present invention is the use of such a transgenic nonhuman animal for obtaining a B cell producing an antibody of interest or for optimizing the binding affinity of an antibody for its target antigen.

A method of optimizing the binding affinity of an antibody variable region is also provided. This can be used to generate high affinity antibodies.

In a particular aspect of the invention, the methods and animals of the invention are used to obtain or design an antibody that is different (as concerns the heavy chain) in sequence from and yet functionally related to a lead antibody of which the heavy and light chain variable are encoded by said rearranged V_{H}DJ_{H} and rearranged VJ segments, respectively. The invention therefore also encompasses methods for modifying a lead antibody antigen binding region or preparing a modified antibody based on a lead antibody. The obtained antibody sequences can include diverse sequences in the complementary determining regions (CDRs) and/or humanized frameworks (FRs) of a non-human antibody in a selective manner to produce an antibody having improved affinity for a target antigen.

The invention provides methods for obtaining a high affinity antibody exhibiting selective binding affinity to a target antigen, or a functional fragment thereof, comprising one or more CDRs having at least one amino acid substitution in one or more CDRs of a lead antibody or lead sequence heavy chain variable region polypeptide, said antibody or functional fragment thereof having target antigen binding activity, target antigen binding specificity or target antigen-inhibitory activity, wherein the target antigen binding affinity of said high affinity antibody is higher affinity relative to parental lead antibody or antibody comprising the lead sequence. The method comprises providing a “Progeny animal” comprising a rearranged V_{H}DJ_{H} and rearranged VJ segment encoding a lead sequence or lead antibody in its germline DNA upstream of the μ constant region, preferably upstream of a Sμ switch, immunizing said animal with target antigen in such a manner suitable to induce B cell mediated affinity maturation (somatic hypermutation) of the lead sequence or lead antibody, and recovering a B cell capable of producing an antibody having target antigen binding activity, target antigen binding specificity or target antigen-inhibitory activity, wherein the target antigen binding affinity of said high affinity antibody is higher affinity relative to parental lead antibody or antibody comprising said rearranged V_{H}DJ_{H} and rearranged VJ segment used as the lead sequence.

Further preferred embodiments are as follows:

In one aspect the invention provides a method for obtaining or producing an antibody of interest binding to a antigen to which a human, non-human, chimeric or humanized lead antibody is specific or a cell producing such antibody, the method comprising:

a) constructing a first non-human animal comprising a sequence encoding at least a rearranged variable region of a heavy chain of a human, non-human, chimeric or humanized lead antibody operably linked to germline or modified heavy chain constant region sequences;

b) constructing a second non-human animal comprising a sequence encoding at least the rearranged variable region of a light chain of a particular human, non-human, chimeric or humanized lead antibody operably linked to germline or modified light chain constant region sequences; and

c) mating animals a) and b) to obtain a progeny animal, and determining whether a B cell of said progeny animal is capable of producing the antibody of interest. Preferably said step of determining whether the progeny animal is capable of producing the antibody of interest comprises determining whether an antibody produced by B cells specifically binds to the antigen to which the human, non-human chimeric or humanized lead antibody is specific. In any of the embodiments, the method may also comprise: treating the progeny animal having the desired phenotype in order to induce somatic hypermutation of the light chain and heavy chain variable region segments and thus the affinity maturation of an antibody produced by B cells from said animal. In any of the foregoing embodiments, the method may also comprise comprising: treating the progeny animal having the desired phenotype in order to stimulate the clonal expansion of the B-cells producing the human, non-human, chimeric or humanized antibody and/or cause an isotype switch from IgM production to the production of IgG antibodies of a desired subtype. Preferably in any of the foregoing embodiments, the method further comprises selecting or isolating a B-cell from said animal which produces the antibody of interest. Preferably, the method comprises selecting a B cell comprises assessing level of antibody production by the B cell. Preferably, said B-cell line is rendered immortal, optionally by fusing said B-cell to a myeloma cell to produce a hybridoma.

In another embodiment, the invention provides a non-human animal having placed in its germline DNA at least:
a sequence encoding at least a rearranged variable region of a heavy chain of a human, non-human, chimeric or humanized lead antibody operably linked to germline or modified heavy chain constant region sequences; and
a sequence encoding at least the rearranged variable region of a light chain of a particular human, non-human, chimeric or humanized lead antibody operably linked to germline or modified light chain constant region sequences.

In another embodiment, the invention provides a non-human animal having placed in its germline DNA at least: a rearranged variable region of a heavy chain of a human, non-human, chimeric or humanized lead antibody upstream of a native μ constant region, and a sequence encoding a heavy chain constant region (i) replacing the native germline DNA that encodes one or more of the native heavy chain constant regions and (ii) operably linked to a switch sequence. Preferably this animal further comprises in its germline DNA a rearranged variable region of an immunoglobulin light chain of a human, non-human, chimeric or humanized lead antibody.

In another embodiment, the invention provides a set of vectors suitable for use as targeting constructs comprising:
a first vector comprising a sequence encoding at least a rearranged variable region of a heavy chain of a human, non-human, chimeric or humanized lead antibody operably linked to germline or modified heavy chain constant region sequences; and
a second vector comprising a sequence encoding at least the rearranged variable region of a light chain of a particular human, non-human, chimeric or humanized lead antibody operably linked to germline or modified light chain constant region sequences.

In another embodiment, the invention provides a vector suitable for use as a targeting construct comprising at least a portion of an IgH locus, said vector or construct further comprising:
a rearranged variable region of heavy chain of a human, non-human, chimeric or humanized lead antibody upstream of a μ constant region, and
a sequence encoding a heavy chain constant region (i) replacing the native DNA that encodes one or more of the native heavy chain constant regions in said IgH locus and (ii) operably linked to a switch sequence. In another embodiment, the invention provides a set of vectors suitable for use as a targeting construct comprising: a first vector as described in the preceding sentence; and
a second vector comprising a sequence encoding at least the rearranged variable region of a light chain of a particular human, non-human, chimeric or humanized lead antibody operably linked to germ line or modified constant region sequences.

In another embodiment, the invention provides an isotype switched cell having integrated in its DNA at least:
a sequence encoding at least a rearranged variable region of a heavy chain of a non-human, chimeric or humanized lead antibody operably linked to germline or modified constant region sequences; and
a sequence encoding at least the rearranged variable region of a light chain of a particular non-human, chimeric or humanized lead antibody operably linked to germline or modified constant region sequences,
wherein said cell has undergone isotype switching.

In another embodiment, the invention provides a non-human B cell having integrated in its DNA at least:
a sequence encoding at least a rearranged variable region of a heavy chain of a non-human, chimeric or humanized lead antibody operably linked to germline or modified constant region sequences; and
a sequence encoding at least the rearranged variable region of a light chain of a particular non-human, chimeric or humanized lead antibody operably linked to germline or modified constant region sequences,
wherein said cell expresses a single antibody species.

In another embodiment, the invention provides a non-human B cell having integrated in its DNA at least:
a sequence encoding at least a rearranged variable region of a heavy chain of a non-human, chimeric or humanized lead antibody operably linked to germline or modified constant region sequences; and
a sequence encoding at least the rearranged variable region of a light chain of a particular non-human, chimeric or humanized lead antibody operably linked to germ line or modified constant region sequences,
wherein said cell does not contain in its genomic DNA sequences capable of giving rise to an antibody different in its variable region sequence from that encoded by said rearranged variable region sequences.

A number of further preferred embodiments are described herein, which the person of skill will appreciate can be applied to any of the embodiments of the methods, animals, vectors or cells described herein. In one aspect of the method, animal, vector or cells herein, said sequences encoding a rearranged variable region of a heavy chain and rearranged variable region of a light chain are independently expressed by the cell, and preferably expressed under the control of native (to the species of origin of the cell) or optionally non-native (e.g. human) immunoglobulin regulatory sequences. In another aspect, said rearranged variable region of a heavy chain and/or light chain is derived from a human lead antibody. In another aspect, said rearranged variable region of a heavy chain and/or light chain is derived from a non-human lead antibody. In another aspect, said rearranged variable region of a heavy chain and/or light chain is derived from a murine lead antibody. In another aspect, said rearranged variable region of a heavy chain and/or light chain are derived from a murine lead antibody having one or more amino acid substitutions. In another aspect, said rearranged variable region of a heavy chain and/or light chain is derived from a chimeric lead antibody. In another aspect, said rearranged variable region of a heavy chain and/or light chain is derived from a CDR grafted lead antibody. In yet another aspect, said rearranged variable region of a heavy chain and/or light chain is derived from a lead humanized lead antibody. Preferably said rearranged variable region of a heavy chain or light chain is obtained or derived from a lead antibody of known specificity.

In any of the methods, animals, vectors or cells herein, said heavy chain constant region sequence may be of non-human origin. In any of the methods, animals, vectors or cells herein, said light chain constant region sequence is of non-human origin. In any of the methods, animals, vectors or cells herein, said heavy chain constant region sequence is of murine origin. In any of the methods, animals, vectors or cells herein, said heavy chain constant region sequence is of human origin. In any of the methods, animals, vectors or cells herein, said light chain constant region sequence is of human origin. In any of the methods, animals, vectors or cells herein, said heavy chain constant region sequence is of the γ isotype, optionally of the G1, G2, G3 or G4 subtype. In a preferred example, said heavy chain constant region is of the G1 subtype and truncated 5′ proximal to the codon coding for the cysteine present in the hinge region and involved in the interchain disulphide bridge, representing a sequence giving rise to a Fab portion. In any of the methods, animals, vectors or cells herein, a constant region sequence is furthermore recombinantly joined to a detectable marker.

In any of the methods, animals, vectors or cells herein, said rearranged variable region of a heavy chain can be placed upstream of a native μ constant region, and a sequence encoding a heavy chain constant region (i) replaces the native DNA that encodes one or more of the native heavy chain constant regions and (ii) is operably linked to a switch sequence.

In any of the methods, animals, vectors or cells herein, said constant region sequences comprise a heavy chain constant region replacing a murine a region, the murine Cγ3, Cγ1, Cγ2b and Cγ2a region set, and/or the ε heavy chain constant region.

In a preferred embodiment, said constant region sequences comprise a human ε or γ heavy chain constant region sequence replacing a murine ε heavy chain constant region.

In another embodiment, said constant region sequences comprise a human ε or γ heavy chain constant region sequence replacing a murine ε heavy chain constant region and the animal, vector or cell comprises in its DNA an arrangement as follows:
5′-Sε-human Cε-Sα-Cα-3′, or
5′-Sε-human Cγ-Sα-Cα-3′,
wherein C represents a constant region, γ may be any human γ constant region subtype G1, G2, G3 or G4 or portion thereof, S represents a switch sequence, and Sε, Sα and Cα may be of human or non-human origin.

In another embodiment, said constant region sequences comprise a human γ heavy chain constant region sequence replacing a murine γ heavy chain constant region, and the animal, vector or cell comprises in its DNA an arrangement as follows:
5′-Sγ-human Cγ-3′
wherein S represents a switch sequence, Cγ represents a human constant region γ subtype G1, G2, G3 or G4 or portion thereof, and Sγ may be of human or non-human origin.

In another embodiment, said constant region sequences comprise a first heavy chain constant region replacing a first native constant region, and a second heavy chain constant region replacing a second native heavy chain constant region.

In another embodiment, said first heavy chain constant region replaces the murine α region and/or the murine Cγ3, Cγ1, Cγ2b and Cγ2a region set, and said second heavy chain constant region replaces the murine heavy chain constant region.

In another embodiment, a γ heavy chain constant region sequence replaces a murine γ heavy chain constant region, and the animal, vector or cell comprises in its DNA an arrangement as follows:
5′-Sγ-replacement Cγ₁-S(ε or α)-replacement Cγ₂-3′
wherein S represents a switch sequence, Cγ₁ and Cγ₂ each represent a different constant region γ subtype.

In another embodiment, a human γ heavy chain constant region sequence replaces a murine γ heavy chain constant region, and the animal, vector or cell comprises in its DNA an arrangement as follows:
5′-Sγ-human Cγ₁-S(ε or α)-human Cγ₂-3′
wherein S represents a switch sequence, Cγ₁ and Cγ₂ each represent a different human constant region γ subtype independently selected from G1, G2, G3 or G4, and each of Sε, Sα and Sγ may be of human or murine origin.

In another embodiment, a human γ heavy chain constant region sequence replaces a murine γ heavy chain constant region, and the animal comprises in its germline DNA an arrangement as follows:
5′-Sγ3-human Cγ₁-Sε-human Cγ₂-Sα-Cα-3′
wherein S represents a switch sequence, C represents a constant region, Cγ₁ and Cγ₂ represent a human constant region γ subtype independently selected from G1, G2, G3 or G4, and each of Cα, Sε, Sα and Sγ may be of human or murine origin.

In preferred embodiments, Sγ is Sγ3 of murine origin.

In any of the methods, animals, vectors or cells herein, the animal or cell is preferably a rat or mouse, or the cell is a rat or mouse cell.

In preferred embodiment of any of the methods, animals, vectors or cells herein, the B cells of said animal consists essentially of B cells which produce the antibody of interest which binds to an antigen to which the lead antibody is specific. Preferably the B cells express the antibody of interest under the control of native (to the species of origin of the B cell) regulatory sequences.

In another embodiment, the invention provides a method for obtaining an antibody of interest or cell producing it, the method comprising:
providing a non-human animal according to any one of the embodiment described herein; and
treating the progeny animal having the desired phenotype in order to induce somatic hypermutation of the V_{H}DJ_{H} and V_{L}J_{L} segments and thus the affinity maturation of an antibody produced by B cells from said animal.

In another embodiment, the invention provides a method for obtaining an antibody of interest or cell producing it, the method comprising:
providing a non-human animal according to any one of the embodiment described herein; and
treating the progeny animal having the desired phenotype in order to stimulate the clonal expansion of the B-cells producing the antibody and/or cause a class switch from IgM production to the production of IgG antibodies of a desired subtype.

In preferred embodiments of the foregoing methods, the methods further comprise:
selecting a B-cell from said animal which encodes or produces an antibody of interest, wherein said antibody of interest binds the same antigen as the antibody from which the lead antibody sequence was derived. In another embodiment of any of the methods, the method further comprises assessing level of antibody production by the B cell.

In another embodiment of any of the methods, the method further comprises rendering said B-cell line immortal, optionally, further comprising fusing said B-cell to a myeloma cell to produce a hybridoma.

In another embodiment, the invention provides a B cell obtained from a non-human animal of any of the embodiments herein, or according to any methods herein. Also encompassed is a cell obtained by immortalizing a B cell so obtained, including but not limited to a hybridoma obtained by fusing said B cell with a second cell. Also encompassed are antibodies produced by any of the cells of the invention, optionally wherein said antibody is a Fab fragment.

In some embodiment the invention further comprise an antibody obtained according to the present embodiment having a glycosylation distinguishable from an antibody of the same amino acid sequence expressed in a murine host cell. Said antibody may have decreased (or absent) fucose content in N-acetylglucosamine of the reducing terminal of an N-glycoside-linked sugar chain compared to an antibody of the same amino acid sequence expressed in a murine host cell, or where and/or increased ability to induce ADCC activity toward a cell expressing an antigen for which the antibody is specific.

In another embodiment, the invention provides cell according to any of the embodiments herein, wherein said cell secretes said antibody of interest into an extracellular medium when maintained in culture. Preferably said cell secretes solely said antibody of interest.

In any of the embodiments, herein the rearranged variable region of an immunoglobulin heavy chain is a rearranged V_{H}DJ_{H} portion and/or the rearranged variable region of an immunoglobulin light chain is a rearranged V-J portion.

In other embodiment, the invention provides a method for producing a functional antibody comprising a heavy chain and a light chain, which comprises the steps of:
maintaining the cell of any of the embodiments herein in a nutrient medium, so that the cell expresses said rearranged variable region of a heavy chain and said rearranged variable region of a light chain and the resultant chains are intracellularly assembled together to form the immunoglobulin which is then secreted in a form capable of specifically binding to antigen to which the lead antibody is specific. Optionally the method further comprises recovering said antibody.

### BRIEF DESCRIPTION OF THE FIGURES

FIGS. 1 and 2 are schematic diagrams for the construction of “light chain only” and “heavy chain only” mice. Shown in FIG. 1 are the constructs for the “Light chain only animals”, a targeting vector that comprises as starting point a portion of the murine CK locus from J region to the constant region gene CK. This starting is construct modified using the elements as described, by substituting by homologous recombination, the mouse CK exons with the human CK exons, and by inserting by homologous recombination human light chain V-J sequences upstream of the constant region CK exons. The murine regulatory sequences are retained. The targeting vector comprises sequences flanking the aforementioned elements which will allow targeted homologous recombination in the germline locus of a mouse ES cell.

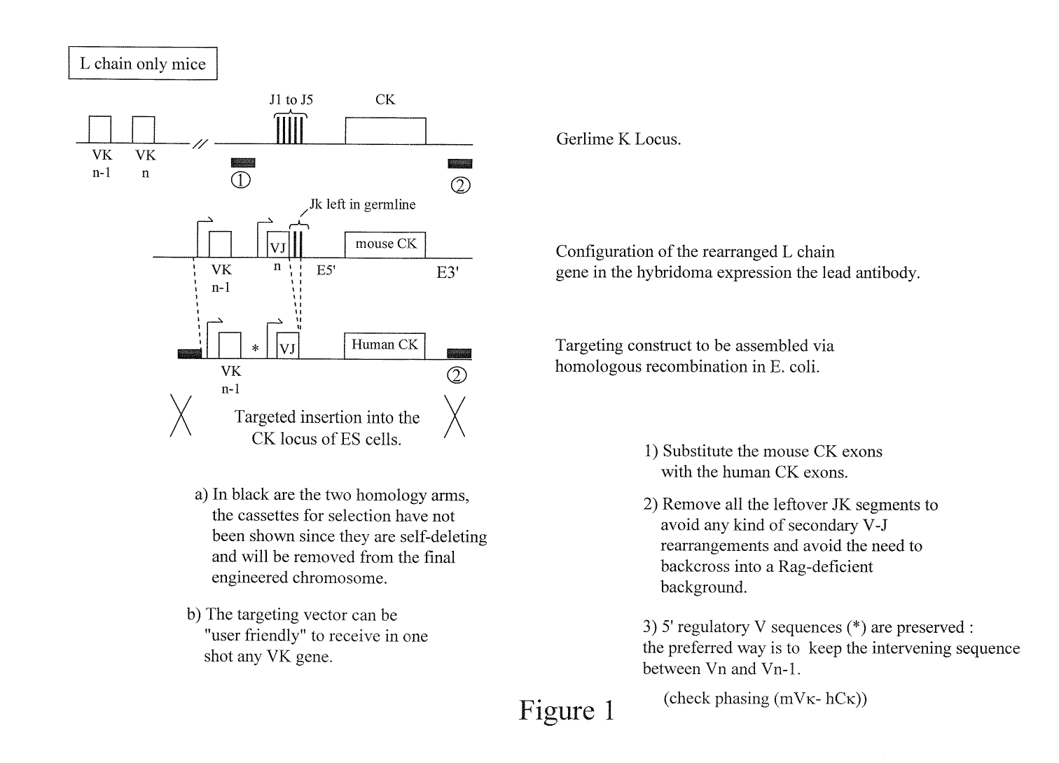

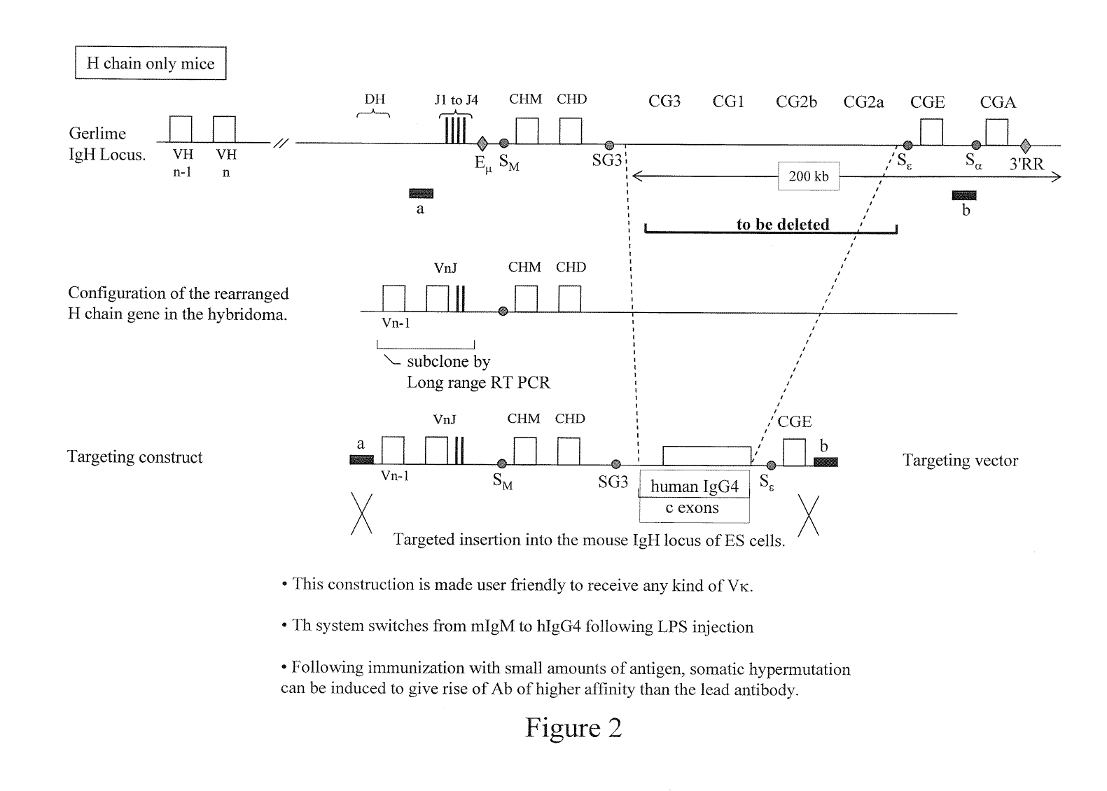

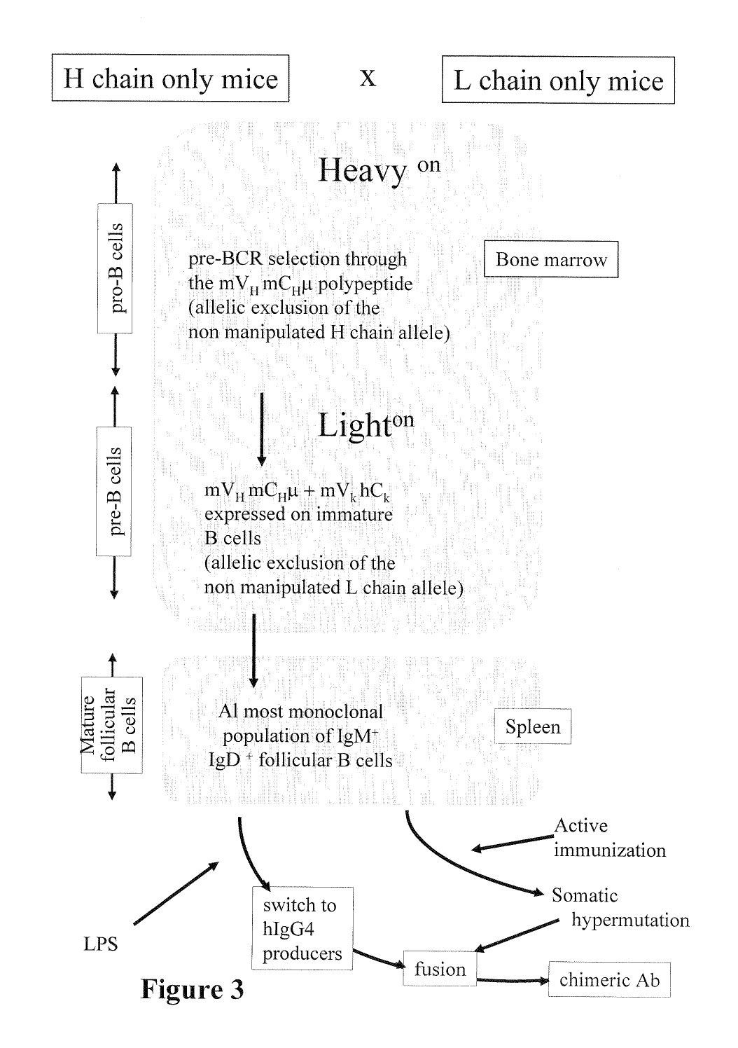

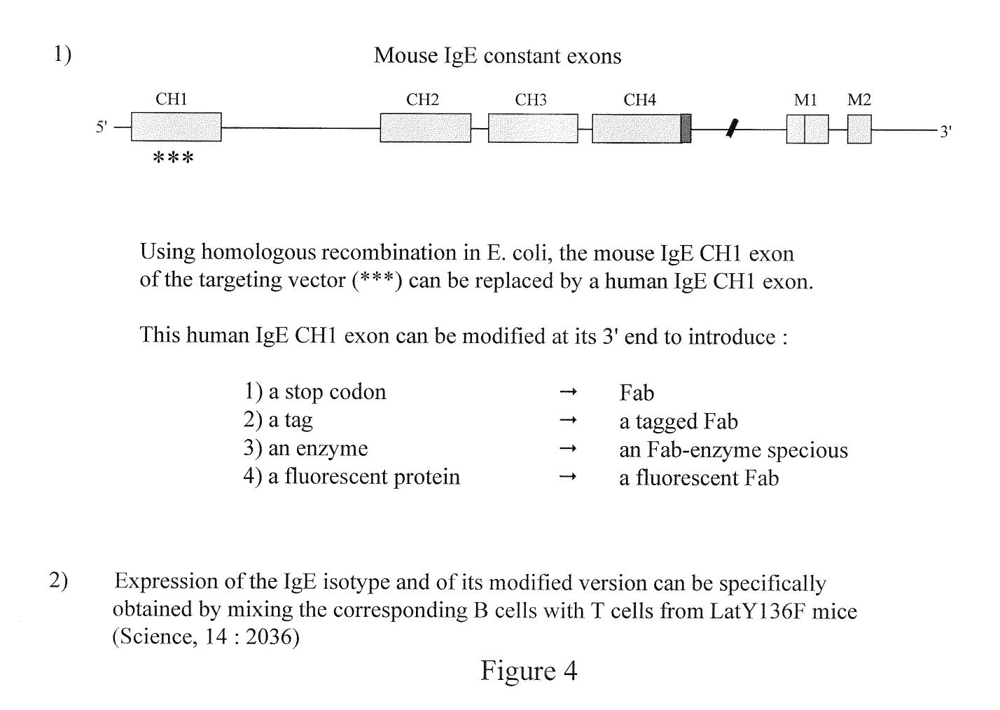

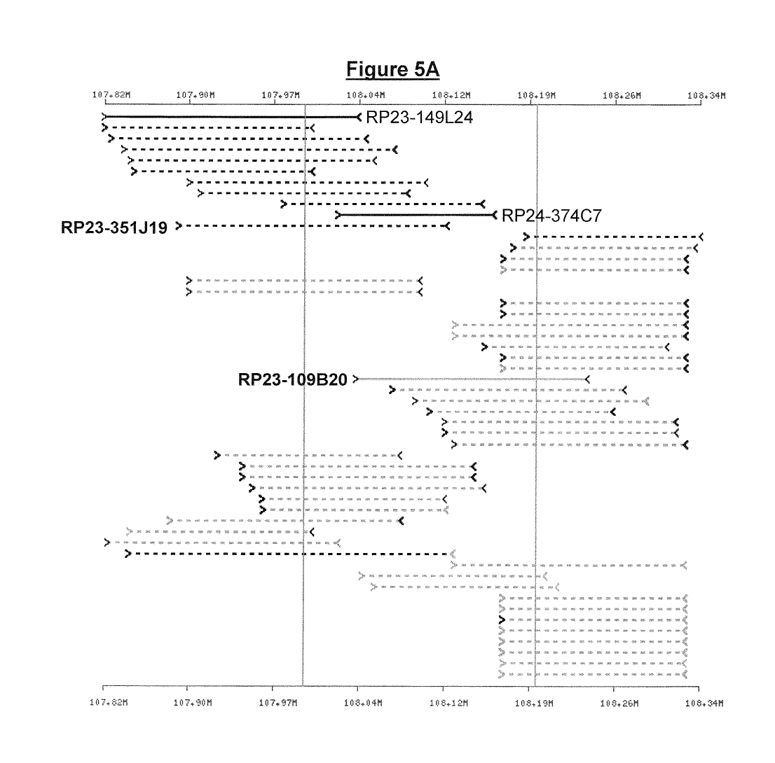

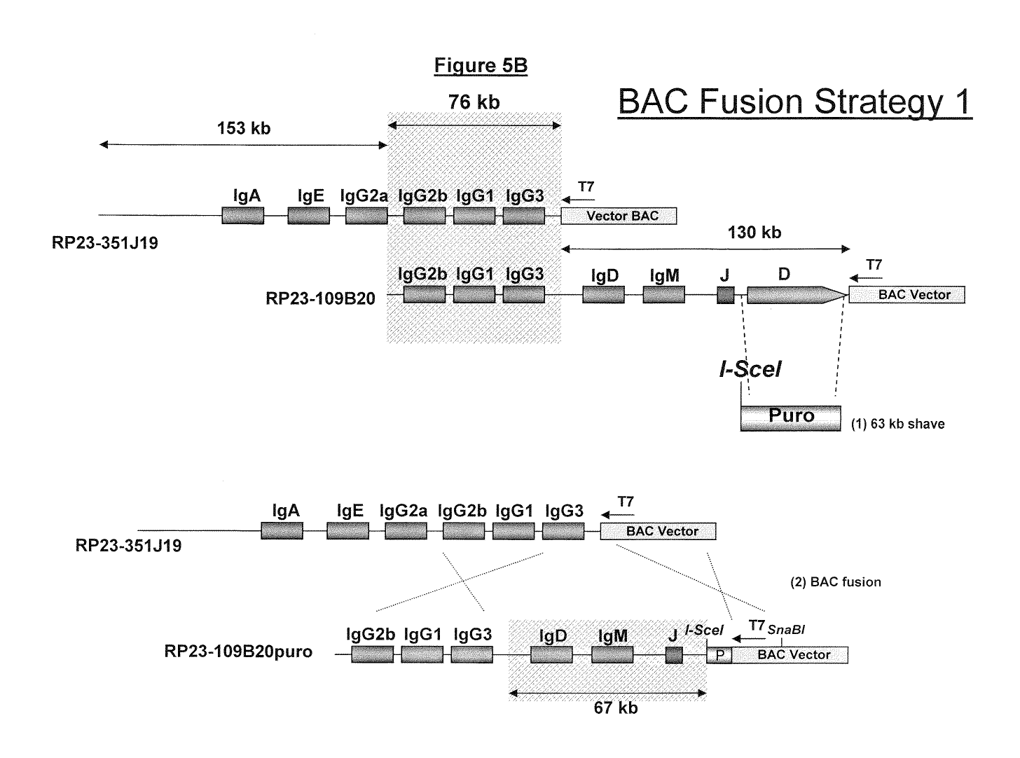

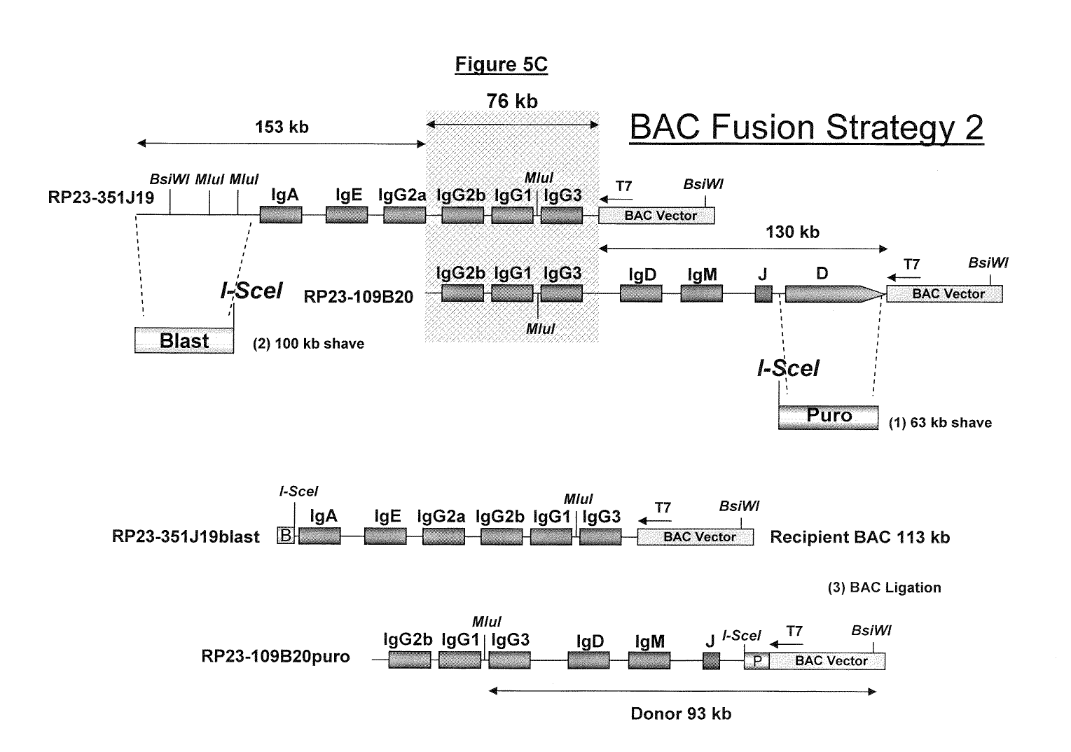

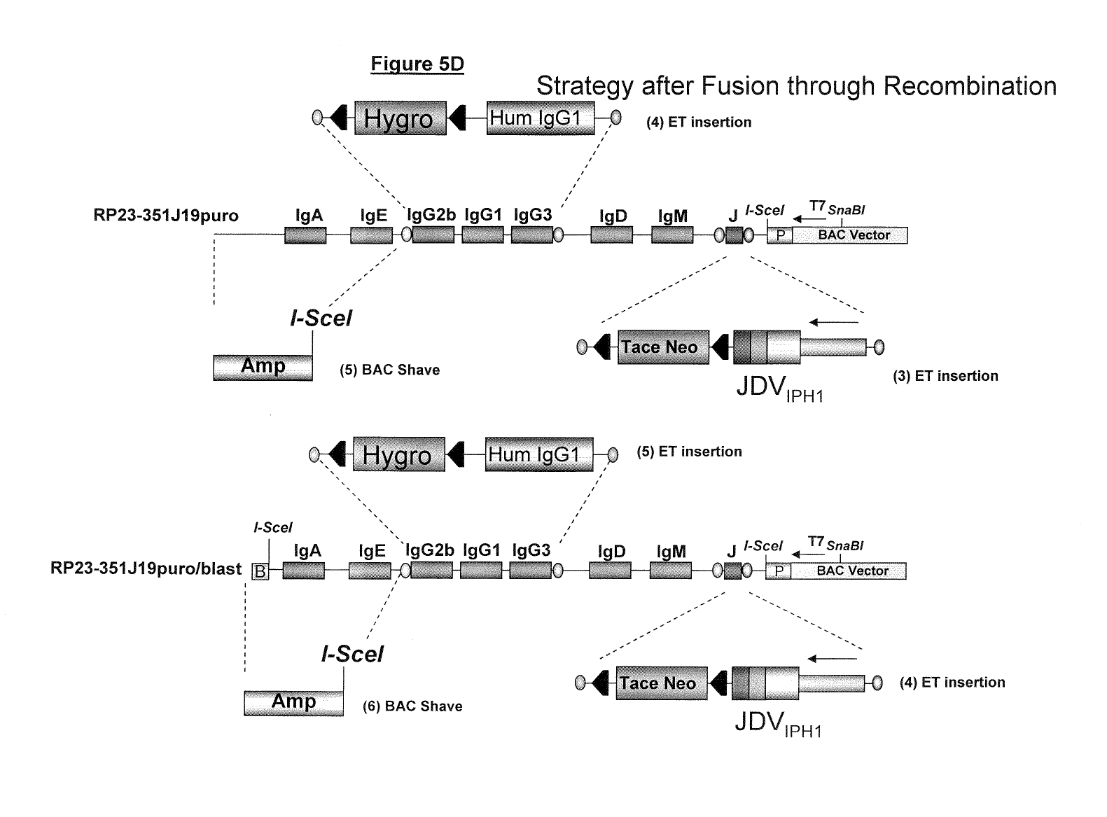

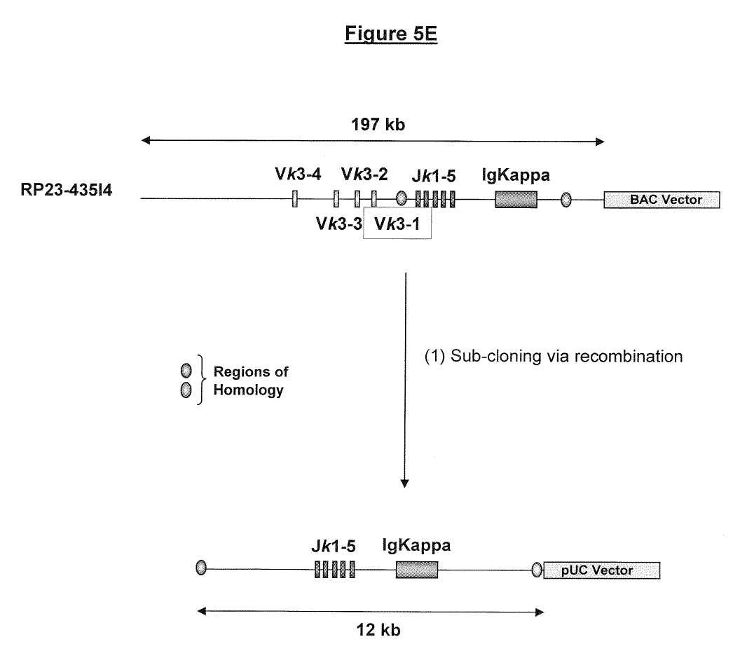

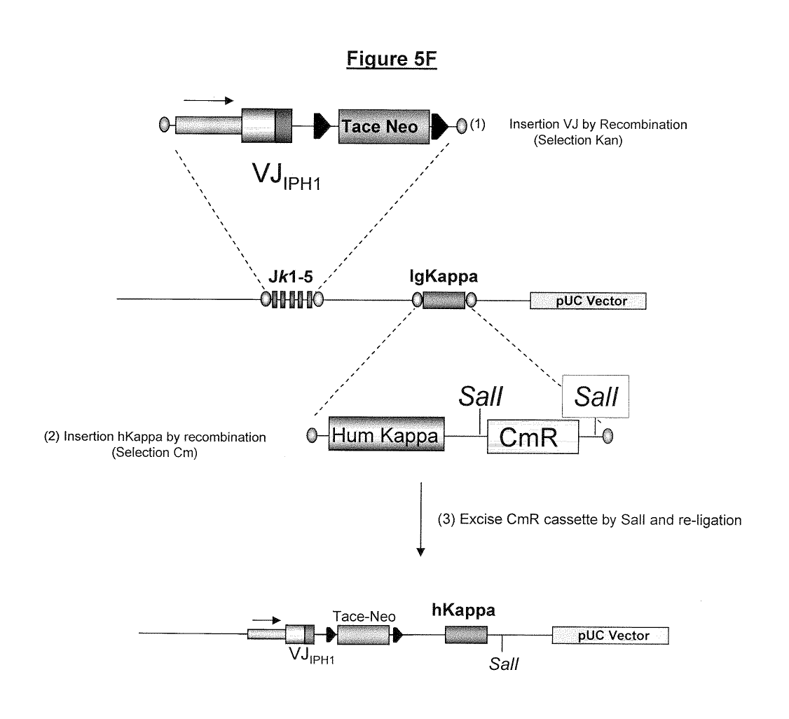

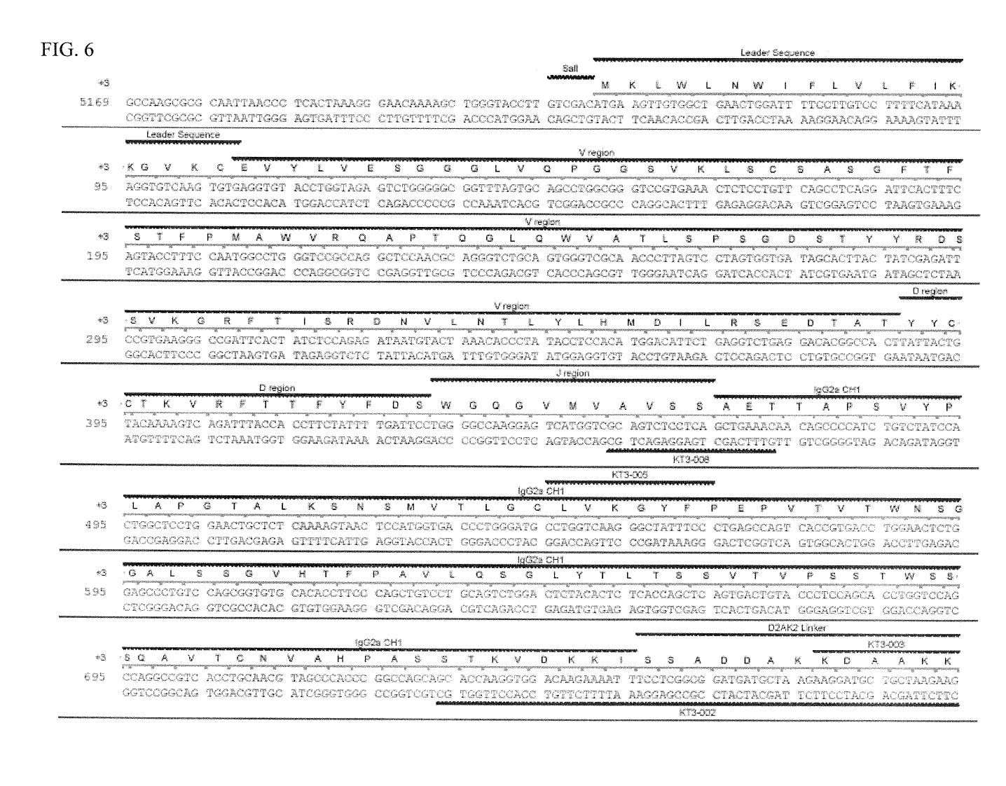

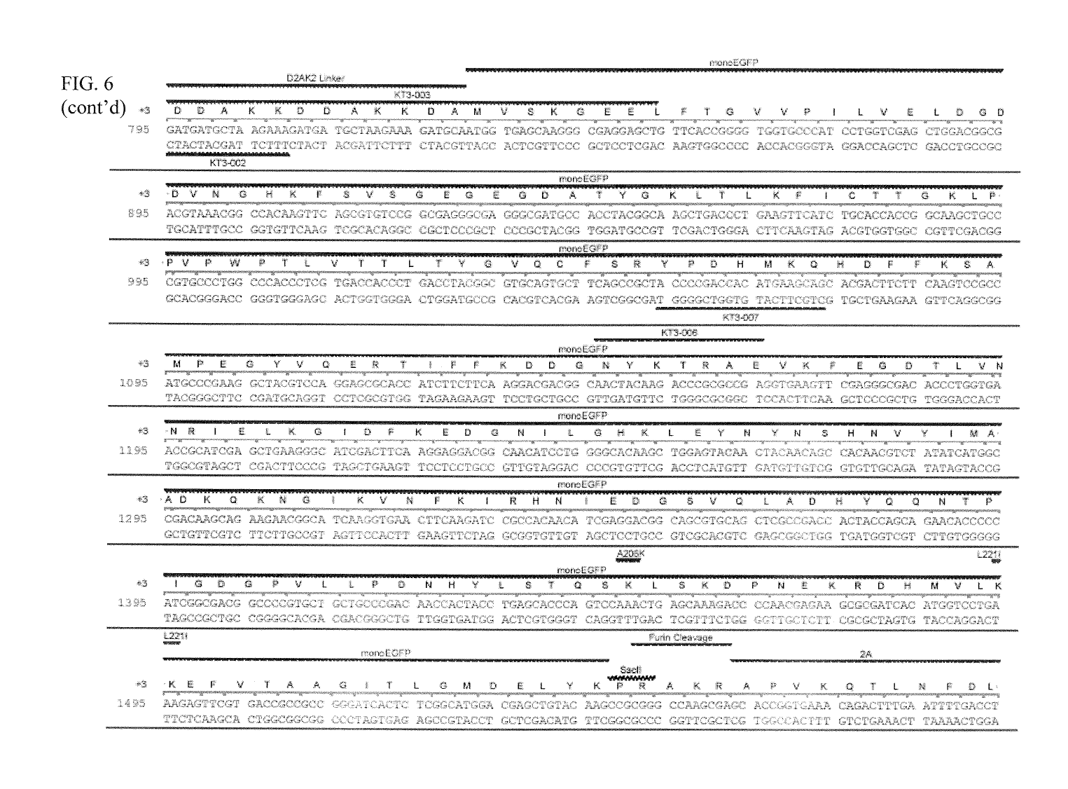

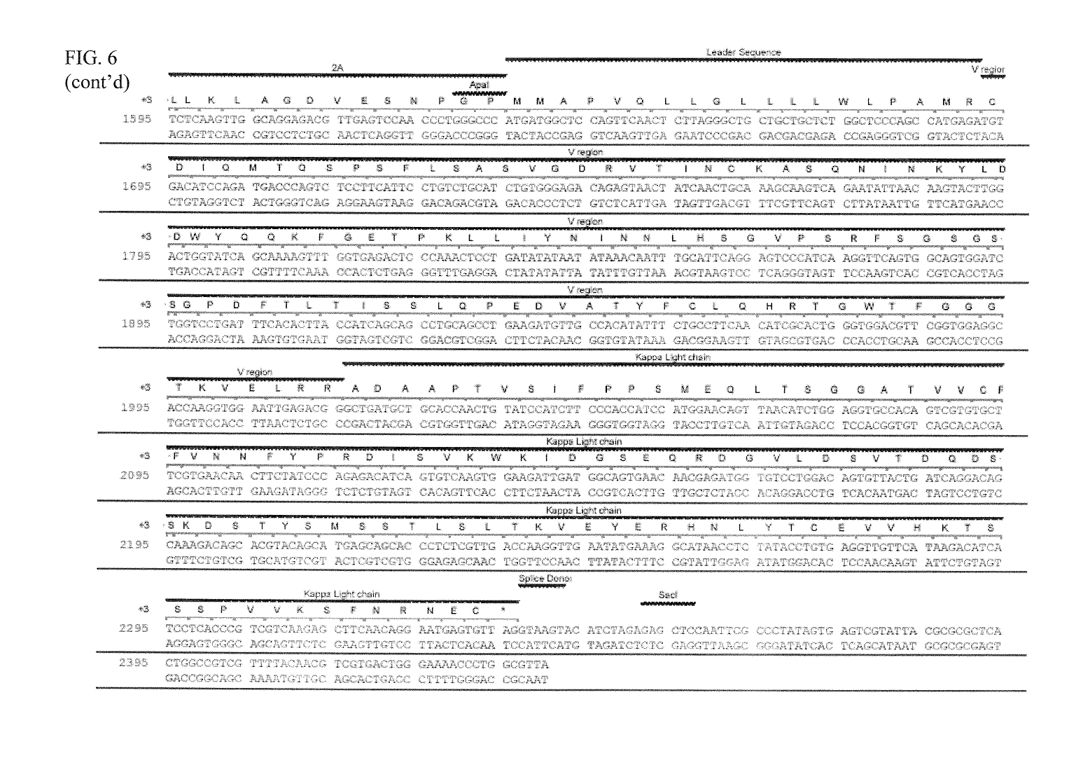

Shown in FIG. 2 are “Heavy chain only animals” constructed with the use of a targeting vector that comprises a portion of the murine IgH locus from J region to the constant region genes (e.g. Cε), and modified using the elements as described. The targeting construct comprises a rearranged V_{H}DJ_{H} portion of a selected immunoglobulin heavy chain gene (e.g. from a human, chimeric or humanized lead antibody) placed upstream of the murine μ constant region. A second sequence encoding the human heavy chain constant region G4 is incorporated upstream of the Sα switch (S) sequence and downstream of the Sγ3 switch sequence, replacing the murine germline DNA that encodes the Cγ3, Cγ1, Cγ2b and Cγ2a heavy chain constant region set. The targeting vector comprises sequences flanking the aforementioned elements (e.g. flanking the rearranged V_{H}DJ_{H} portion and the human heavy chain constant region G4) which will allow targeted homologous recombination in the germline locus of a mouse ES cell.

FIG. 3 is a schematic representation for the generation of progeny mice that result from the mating of heavy chain only mice and light chain only mice and that express a human, humanized or chimeric antibody of interest. The figure also illustrates methods for inducing “class switching” of antibodies and affinity maturation of the human, chimeric or humanized antibodies in vivo.

FIG. 4 is a diagram for the construction of a heavy chain only mouse capable of expressing in its B cells an antibody of the IgE isotype having a human heavy chain E constant region. The mouse Cε CH exons are replaced by a human CH exons of a desired isotype. B cells from a progeny animal constructed in this way can be brought into contact with CD4 T cells from a Lat Y136F mutant mouse (preferably by adoptive transfer of the CD4 T cells to the progeny animal or incubation of the CD4 T cells with B cells from the progeny animal)) thereby inducing the expression of antibodies of the IgE isotype.

FIG. 5A shows the overlapping BACs used to engineer the mouse Ig Heavy chain locus, which BACs are subsequently used to generate a fused recombinant BAC containing the D and J gene segments as well as the C genes.

FIG. 5B shows a first strategy to prepare a recombinant BAC containing the D and J gene segments as well as the C genes, where the D gene segment cluster is deleted and replaced with a selectable marker, and overlapping BACs are fused by homologous recombination techniques.

FIG. 5C shows a second strategy to prepare a recombinant BAC containing the D and J gene segments as well as the C genes, where the D gene segment cluster is deleted and replaced with a selectable marker in the first BAC and a selectable marker is introduced to the second BAC, and overlapping BACs are ligated.

FIG. 5D show the BACs RP23-351J19puro and RP23-351J19puro/blast obtained from the steps in FIGS. 5B and 5C, respectively, and the strategy used to substitution of the sequences coding for the mouse IgG2b, IgG1, IgG3c, IgG2a genes by the sequence coding for the human IgG1 C gene wherein (i) a human IgG1 constant (C) gene cassette is constructed and inserted into the BACs by homologous recombination techniques and (ii) a cassette containing the heavy chain variable region gene (VHDHJH^{IPH1}) is constructed and inserted into the BACs by homologous recombination techniques.

FIG. 5E shows the first of three steps for engineering of the mouse Ig C kappa locus, whereby a portion of BAC containing the mouse IgKappa gene is subcloned into a vector.

FIG. 5F shows the second and third of three steps for engineering of the mouse Ig C kappa locus, whereby the vector of FIG. 5E receives (i) a genomic fragment corresponding to the promoter of the VKJK^{IPH1} gene and to the VKJK^{IPH1} gene itself previously isolated from hybridoma IPH1, and (b) human CK gene replacing the mouse CK gene. Both elements are introduced by homologous recombination techniques.

FIG. 6 shows the sequences (SEQ ID NOs: 1 and 2) of the vector used to test the principle of construction of a Fab-linkerEGFP version of the KT3 mAb.

### BRIEF DESCRIPTION OF THE TABLES

Table 1 provides exemplary humanized antibodies suitable for use in the instant invention. The references cited within the Table are incorporated by reference in their entireties, particularly with respect to the nucleic acid and amino acid sequences disclosed therein for each respective human, humanized or chimeric antibody.

Table 2 discloses various exemplary myeloma cells suitable for immortalization of antibody producing B-cells derived from humans, mice and rats. These myeloma cells can be obtained from the American Type Culture Collection, 10801 University Blvd., Manassas, Va. 20110.

Table 3. Commonly used ligand/binding partner systems. Polynucleotides encoding the peptides/polypeptides disclosed in the “Binding Partner” column can be joined, in frame, to the constant regions of polynucleotides encoding the antibody heavy and/or light chains that are used in the preparation of a DNA construct for insertion into an animal.

### DETAILED DESCRIPTION OF THE INVENTION

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., alpha-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acids can be either single stranded or double stranded.

The term “transfection” refers to the introduction of a nucleic acid, e.g., a targeting vector, into a recipient cell by gene transfer.

“Transformation”, as used herein, refers to a process in which a cell's genotype is changed as a result of the cellular uptake of exogenous DNA or RNA.

As used herein, the term “transgene” refers to a nucleic acid sequence which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome at such a position or otherwise in such a way as to alter the genome of the cell into which it is inserted. A transgene can be operably linked to one or more transcriptional regulatory sequences and any other nucleic acid, such as introns, that may be necessary for optimal expression of a selected nucleic acid.

The term “transgenic” is used herein as an adjective to describe the property, for example, of an animal or a construct, of harboring a transgene. For instance, as used herein, a “transgenic organism” is any animal, preferably a non-human mammal, in which one or more of the cells of the animal contain heterologous nucleic acid introduced by way of human intervention, such as by transgenesis techniques well known in the art, including but not limited to replacement of a homologous endogenous gene by homologous recombination. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express an immunoglobulin. The terms “founder line” and “founder animal” refer to those animals that are the mature product of the embryos to which the transgene was added, i.e., those animals that grew from the embryos into which DNA was inserted, and that were implanted into one or more surrogate hosts. The present invention covers such animals as well as any descendents or progeny carrying the herein-described transgene or expression construct.

As used herein, the expressions “cell,” “cell line,” and “cell culture” are used interchangeably and all such designations include progeny. For the purposes of the present invention, such cells can be derived from a transgenic mammal, or produced directly by transformation of cells with one of the herein-described targeting constructs or vectors. The words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as obtained in the originally transformed cell or animal are included.

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under-expressed or not expressed at all.

A nucleic acid is “operably linked” when it is placed into a functional relationship with another nucleic acid sequence. For instance, a promoter or enhancer is operably linked to a coding sequence if it affects the transcription of the sequence. For switch sequences, operably linked indicates that the sequences are capable of effecting switch recombination.

The term “rearranged” refers to a configuration of a heavy chain or light chain immunoglobulin locus wherein a V segment is positioned immediately adjacent to a D-J or J segment in a conformation encoding essentially a complete VH or VL domain, respectively. A rearranged immunoglobulin gene locus can be identified by comparison to germline DNA.

The term “unrearranged” or “germline configuration” in reference to a V segment refers to the configuration wherein the V segment is not recombined so as to he immediately adjacent to a D or J segment.

“Isotype” refers to the antibody class that is encoded by heavy chain constant region genes. Heavy chains are classified as gamma (γ), mu (μ), alpha (α), delta (δ), or epsilon (ε), and define the antibody's isotype as IgG, IgM, IgA, IgD and IgE, respectively. Additional structural variations characterize distinct subtypes of IgG (e.g., IgG1, IgG2, IgG3 and IgG4) and IgA (e.g., IgA1 and IgA2). “Isotype switching” refers to the phenomenon by which the class, or isotype, of an antibody changes from one Ig class to one of the other Ig classes.

“Nonswitched isotype” refers to the isotypic class of heavy chain that is produced when no isotype switching has taken place; the C_{H} gene encoding the nonswitched isotype is typically the first C_{H} gene immediately downstream from the functionally rearranged VDJ gene (for example Cμ in non modified configuration). Isotype switching has been classified as classical or non-classical isotype switching.

The term “switch sequence” refers to those DNA sequences responsible for switch recombination which mediates isotype switching. Switch sequences, switch donor and switch acceptor are further described herein.

The term “high affinity” for an antibody refers to an equilibrium association constant (Ka) of at least about 10⁷M⁻¹, at least about 10⁸M⁻¹, at least about 10⁹M⁻¹ , at least about 10¹⁰M⁻¹ at least about 10¹¹M⁻¹, or at least about 10¹²M⁻¹ or greater, e.g., up to 10¹³M⁻¹ or 10¹⁴M⁻¹ or greater. However, “high affinity” binding can vary for other antibody isotypes.

### Lead Sequence

Any monoclonal antibody known in the art or cell which produces an antibody can serve as a basis for providing the nucleic acids or nucleic acid information necessary for the construction of transgenic animals according to the subject invention. Such an antibody or nucleic acid sequence can also be referred to as a “lead antibody” or “lead sequence”. The “lead antibody” or “lead sequence” will generally comprise a portion of the antibody or sequence encoding such a portion which confers antigen binding ability onto the antibody.

For example, various non-limiting examples of humanized antibodies that have been reported in the literature are provided in Table 1 (each of these references is hereby incorporated by reference in its entirety, particularly with respect to the nucleic acid and amino acid sequences that encode the humanized antibodies disclosed therein). The nucleic acids disclosed within these references can be utilized in the construction of the “light chain only” or “heavy chain only” animals disclosed infra. Generally the lead antibody is a human antibody (for example as can be obtained by immunization of a mouse carrying a human Ig locus), a chimeric antibody, a non-human antibody (e.g. murine), or a humanized antibody. However, in some cases (for example where an increase in affinity for a target antigen is sought) the lead antibody may be a polypeptide obtained by combinatorial (e.g. phage display) techniques.

The terms “immunoglobulin(s)” and “antibody(ies)” may be used interchangeably.

“Chimeric antibody(ies)” are immunoglobulin molecules comprising a human and non-human portion. The chimeric antibody may have the antigen binding specificity of the non-human antibody molecule and the effector function conferred by the human antibody molecule. The term “chimeric antibody(ies)” thus encompasses antibodies in which all or part(s) of the variable region of the antibody molecules are derived from one species of animal and the constant regions of the antibody molecule are derived from a second animal. In certain embodiments of the invention, the constant regions of the antibody are derived from humans and the variable regions of the chimeric antibody can be derived from mice, rats, hamsters, rabbits, chickens, horses, cows, or sheep. Methods of making chimeric antibodies are also well-known in the art (see, for example, U.S. Pat. No. 4,816,567, which is hereby incorporated by reference in its entirety). The term “chimeric antibodies” encompasses humanized and CDR grafted antibodies. It will be appreciated that CDR grafting may involve retaining sequences from all or only from a portion (i.e. at least one) of the CDRs of a donor antibody. It will also be appreciated that CDR grafting may involve retaining the entire CDR sequence or only those resides only the specificity-determining residues (SDRs), the residues that are essential for the surface complementarity of the Ab and its ligand. Moreover, residues may be exchanged to residues having similar properties. Framework, CDR sequences other than the SDRs may originate from a single donor or may be assembled from multiple donor sequences.

The term “humanized antibody(ies)” encompasses antibodies that have been humanized according to methods known in the art (see, for example, U.S. Pat. Nos. 5,585,089; 5,530,101; 5,693,762; 5,693,761; and 5,714,350, each of which is hereby incorporated by reference in its entirety).

In yet other embodiments of the subject invention, transgenic animals can be constructed using nucleic acids that encode human monoclonal antibodies (i.e. where both variable and constant gene segment are from human origin, but may be recombined in another species). In such embodiments, the nucleic acids encoding human monoclonal antibody sequences are utilized in constructing transgenic animals as set forth herein.

Methods for obtaining humanized antibodies suitable for use as a lead antibody are known in the art (see, for example, U.S. Pat. Nos. 5,585,089; 5,530,101; 5,693,762; 5,693,761; and 5,714,350, each of which is hereby incorporated by reference in its entirety, particularly with respect to the methods of making humanized antibodies that are disclosed therein). The structure of a non-human, donor antibody (e.g., a mouse monoclonal antibody) is predicted based on computer modeling and key amino acids in the framework are predicted to be necessary to retain the shape, and thus the binding specificity of the CDRs. These few key murine donor amino acids are selected based on their positions and characters with in a few defined categories and substituted into a human acceptor antibody framework along with the donor CDRs. For example, category 1: the amino acid position is in a CDR as defined by Kabat et al. Kabat and Wu (1972) Proc. Natl. Acad. Sci. USA 69: 960; category 2: if an amino acid in the framework of the human acceptor immunoglobulin is unusual, and if the donor amino acid at that position is typical for human sequences, then the donor amino acid rather than the acceptor many be selected; category 3: in the position immediately adjacent to one or more of the 3 CDR's in the primary sequence of the humanized immunoglobulin chain, the donor amino acid(s) rather than the acceptor amino acid may be selected. Based on these criteria, a series of selections of individual amino acids from the donor antibody is conducted. The resulting humanized antibody usually includes about 90% human sequence. The humanized antibody designed by computer modeling is tested for antigen binding. Alternatively, the manufacture of a humanized antibody of a desired specificity can be performed by various commercial sources, such as Aeres Biomedical, Ltd. (London, England).

Methods for obtaining human antibodies are also well known in the art. For example, human antibodies can be obtained by immunizing a mouse carrying a human Ig locus with an antigen of interest. Methods and transgenic mouse for producing human antibodies are described in U.S. Pat. Nos. 6,713,610; 6,673,986; 6,657,103; 6,162,963; 5,939,598; 5,770,429; 6,255,458; 5,877,397; 5,874,299; and International Patent publication nos. WO 99/45962; WO 98/24884; WO 97/13852; WO 94/25585; WO 93/12227; WO 92/03918, the disclosures of all of which are incorporated herein by reference.

The nucleic acids or nucleic acid information necessary for the construction of transgenic animals according to the subject invention can he used in accordance with the invention in any suitable manner.

For the purposes of this invention, the terms “animal” or “animals” includes any non-human animal from which a monoclonal antibody can be made. In particular, non-human animal is a laboratory animal, e.g. mice, rats, hamsters, rabbits, chickens, horses, cows, or sheep. In a preferred embodiment, the non-human animal is a laboratory rodent, e.g. mice, rats, hamsters, etc. While reference is often made within the specification to mice, it will be appreciated that other suitable animals can be used in the same way. Non-limiting examples of suitable animals for the construction of transgenic animals are: rodents (e.g., mice, rats, hamsters, etc.); rabbits; chickens; horses; cows; or sheep.

### Constructing Transgenic Animals

In one aspect of the subject invention, a “light (L) chain only animal” is provided. Such an animal comprises a sequence that encodes at least the rearranged light chain of a lead antibody. The lead antibody is preferably a human, humanized or chimeric antibody, or a portion thereof. For the purpose of this invention, the C_{L}κ sequences are often taken as reference but it is appreciated that C_{L}λ sequences can be used in the same way.

For a “light (L) chain only animal”, the sequence encoding the lead antibody light chain (or portion thereof; e.g., nucleic acids encoding the variable region of a chosen human, humanized or chimeric antibody molecule or a rearranged V-J segment of a chosen antibody) is inserted by homologous recombination into and preferably upstream of a normal or modified mouse C_{L}κ or C_{L}λ sequence. The mouse C_{L}κ or C_{L}λ sequences may for example have been modified to encode human C_{L}κ or C_{L}λ sequences, and may include regulatory elements from human or murine origin (at least enhancer sequences). If desired, the remaining JK segments in the C_{L}κ or C_{L}λ locus can be removed to avoid the possibility of secondary V-J rearrangements and the possible need to backcross animals into an appropriate background (e.g., a Rag-deficient background). Such modified C_{L}κ or C_{L}λ sequences can be engineered in E. coli for example, by homologous recombination. A preferred method of the invention includes the transfer of the modified mouse C_{L}κ or C_{L}λ locus containing a rearranged variable region and modified (preferably to contain human sequences) C_{L}κ or C_{L}λ sequences to ES cells by homologous recombination. After ES cells have been manipulated as described and selected, the ES cells are injected into the inner cell mass (ICM) of blastocysts. Embryos are then transferred into female animals and allowed to mature. Alternatively the modified locus can be transferred to the mice by transgenesis. Further details are provided herein.

The sequence encoding the light chain of a human, humanized or chimeric antibody molecule (or portion thereof) can further comprise additional elements as are set forth infra.

Heavy (H) Chain only Animals

The subject invention also provides a “heavy (H) chain only animal”. Such an animal comprises a sequence that encodes at least the rearranged heavy chain of a lead antibody, preferably a human, humanized or chimeric antibody or a portion thereof (e.g., the variable region of the heavy chain). The sequence is inserted by homologous recombination into a normal or modified non-human animal (e.g. mouse) C_{H} locus. The mouse C_{H} locus may optionally have been modified to encode human C_{H} sequences but includes at least regulatory elements of human or murine origins (at least enhancer sequences) to which the rearranged heavy chain of a lead antibody is operably linked. Such a modified heavy chain locus can be engineered for example in E. coli by homologous recombination. The constant region may be a modified (with respect to the lead antibody) constant region gene, wherein the constant region is different in sequence, species of origin and/or subtype from that of the lead antibody human constant region. For example, a rearranged V_{H}DJ_{H} portion of a selected human, humanized or chimeric antibody heavy chain gene is placed into the germline locus of the mouse ES cell by homologous recombination. A preferred method of the invention comprises the insertion of the modified C_{H} locus containing rearranged variable chain of known lead antibody and a human constant region gene into the heavy chain locus of embryonic stem (ES) cells by homologous recombination. Methods for performing such insertions are well known in the art (see, for example, Lopez-Macia et al., J. Exp. Med. 1999, 189:1791-1798 and Cascalho et al., Science 1996, 272:1649-1652, each which is hereby incorporated by reference in its entirety, particularly with respect to the making of transgenic mice). After ES cells have been manipulated as described and selected, the ES cells are injected into the inner cell mass (ICM) of blastocysts. Embryos are then transferred into female animals and allowed to mature. Alternatively the modified locus can be transferred into a nonhuman animal (e.g. a mouse) by transgenesis. Further details are provided herein.

The sequence encoding the humanized chain of a human, humanized or chimeric lead antibody (or portion thereof) can further comprise additional elements as are set forth infra.

### HCOA2 Animals

In another embodiment, the “heavy chain only animals” are provided that contain a rearranged V_{H}DJ_{H} portion of a selected heavy chain placed, upstream of the murine μ constant region, into the germline locus of the animal (e.g. a mouse). A second sequence encoding a human heavy chain constant region (for example a constant region of G₄ or G1 subtype) is also incorporated into the germline locus of the animal. The sequences are preferably placed into the germline locus of murine ES cells, by homologous recombination, to replace the murine a region, the murine Cγ3, Cγ1, Cγ2b and Cγ2a region set, and/or the murine c heavy chain constant region. “Heavy chain only animals” made in this embodiment of the invention may be referred to as HCOA2 animals.

In another embodiment, two sequences encoding human constant regions, for example human constant regions of the G1 and G4 subtypes, are incorporated by homologous recombination in the mouse locus. One of them is used to replace murine germline sequence encoding either the a region or the murine Cγ3, Cγ1, Cγ2b and Cγ2a region set, and the other is used to replace the mouse c heavy chain constant region, these animals being referred to herein as “HCOA3 animals”. In yet another embodiment, one of the human sequence encodes a modified (preferably human) constant region gene, wherein the constant region is different in sequence, species of origin and/or subtype from that of the lead antibody.

It will be appreciated that the human heavy chain constant region can be arranged in the germline locus of the ES cell in any of a number of suitable manners and configurations. In one example, the human heavy chain constant region sequence is made contiguous with the rearranged V_{H}DJ_{H} portion sequence such that HCOA2 animals express heavy chains having a variable region encoded by the rearranged V_{H}DJ_{H} and a human constant region of the desired isotype transcribed as a single mRNA molecule (e.g. V_{H}DJ_{H}C_{H}). B cells from such animals will not undergo normal development and the heavy chain coding sequences will not be capable of undergoing somatic hypermutation that would modify the heavy chain coding or amino acid sequence. In another example, a human Cμ and/or Cδ heavy chain constant region replaces the murine germline DNA that encodes Cμ and Cδ constant regions. In another example, murine Cμ and Cδ genes remain functional in the HCOA2, HCOA3 and other animals of the invention. Preferably, the animals of the invention are capable of undergoing somatic hypermutation of the human heavy chain coding sequences. In a preferred example, the human heavy chain constant region is used to replace the murine germline DNA that encodes the a region, the Cγ3, Cγ1, Cγ2b and Cγ2a region set, and/or the c heavy chain constant region.

Preferably, the human heavy chain constant region replaces the murine germline DNA that encodes the murine α region, the Cγ3, Cγ1, Cγ2b and Cγ2a region set, and/or the ε heavy chain constant region such that the murine switch sequence upstream of the replaced region(s) deleted remains functional. The human heavy chain constant region is thus placed downstream from or operably linked to a switch sequence, for example a Sγ3 sequence. These animals will express heavy chains having a variable region encoded by the rearranged V_{H}DJ_{H} and upon stimulation to induce a class switch (e.g. with LPS for animals with a Sγ3 sequence) to a human constant region of the desired isotype. This invention thus provides methods whereby the gene segment to be inserted into transgenic animal's genome contains sequences that effectuate isotype switching, so that the heterologous immunoglobulins produced in the transgenic animal and monoclonal antibody clones derived from the B-cells of said animal may be of the desired isotype(s), more particularly of a desired human constant region subtype. Yet more preferably, as further described herein, the transgene is also configured such that the transgenic animal remains able to effect somatic hypermutation of the rearranged V_{H}DJ_{H} portion.

Switch sequences of human or nonhuman (e.g. murine) origin may be grafted from various constant region genes and ligated to other constant region (C_{H}) genes in a construct of the invention used to generate the heavy chain only animals; such grafted switch sequences will typically function independently of the associated C_{H} gene so that switching in the construct will typically be a function of the origin of the associated switch regions. Further references and configurations on switch sequences and constant region regions are provided herein.

The switch sequence and the human heavy chain constant region can generally be arranged in any suitable configuration. At least one of the murine constant region isotypes genes will be functionally replaced with a human constant region gene, e.g. Cμ, Cδ, Cγ, Cα or Cε. If the a murine Cγ region is to be replaced, then preferably the entire Cγ3, Cγ1, Cγ2b and Cγ2a region set is replaced. Heavy chains are classified as γ, μ, α, δ or ε, and define the antibody's isotype as IgG, IgM, IgA, IgD and IgE, respectively. Additional structural variations characterize distinct subtypes of IgG (e.g., IgG1, IgG2, IgG3 and IgG4) and IgA (e.g., IgA1 and IgA2). The transgenic human gene may be the counterpart to the native (e.g. murine) gene which it replaces, e.g. Cγ1→Cγ1, or may of be a different isotype. Preferably, the replaced host region will be other than Cμ and/or other than Cδ. Of particular interest are the a and γ constant regions, which may be interchanged, e.g. Cγ1→Cα; Cγ2→Cα; Cγ3→Cα; Cγ4→Cα; Cα→Cγ1, etc.; Cγ1→Cε, etc.; Cα→Cε, and the like. As mentioned, in preferred embodiments the transgenic animals of the invention have native (e.g. murine) Cμ and Cδ elements and are able to effect in vivo affinity maturation of a rearranged antibody gene and class switch to whichever transgenic human C region, e.g. Cγ, Cα, Cδ or Cε, has been inserted in the nonhuman animal.

In a further example, at least a first and a second human heavy chain constant regions replace the murine germline DNA that encodes the α region, the Cγ3, Cγ1, Cγ2b and Cγ2a region set, and/or the ε heavy chain constant region. This will permit, depending on the method used to induce class switching, more than one antibody format to be produced by cells from an animal. For example, it may be useful to prepare antibodies of different subtypes (e.g. IgG1 and IgG3, IgG1 and IgG4, or IgG2 and IgG4) based on the same lead sequence variable region for purposes of comparing effector function, or to prepare antibodies of a given subtype and Fab fragments thereof to compare pharmacodynamic properties of the resulting antibodies. These heavy chain constant regions can be any isotype or derivative or variant thereof, a sequence encoding a portion thereof (e.g. Fab fragment missing the portion of the heavy chain constant region that would be below the disulfide linkages in the hinge region), or a constant region so modified to have modified (increased or decreased) effector function (see FIG. 4). One example of the latter are constant regions comprising one or more amino acid modifications that increase or decrease Fcγ receptor binding (see below). Preferably each of these human heavy chain constant regions is operably linked to a distinct switch such that the expression can be controlled whereby a transgenic progeny animal according to the invention has B cells producing at a given moment a single particular human heavy chain constant region.

The switch used in the targeting constructs of the invention can be native to the species of animal that is made transgenic, or can be of a different origin. A switch for use in constructing a transgenic mouse may be for example of human or murine origin. Preferably, however, the switch will of murine origin so as to provide optimal functionality in the mouse.

Exemplary Heavy Chain only Animal Targeting Construct

In one example of a heavy chain only animal where a human γ heavy chain constant region sequence replaces a murine γ heavy chain constant region, an animal comprising an arrangement as follows in its germline DNA can be constructed:
5′-Sγ-human Cγ-3′
   wherein S represents a switch sequence, Cγ represents a human constant region γ subtype G1, G2, G3 or G4 or portion thereof and may be different or the same, and Sγ may be of human or non-human (e.g. murine) origin. Most preferably Sγ is Sγ3.

In another example of a heavy chain only animal where a human γ heavy chain constant region sequence replaces a murine γ heavy chain constant region, an animal comprising an arrangement as follows in its germline DNA can be constructed:
5′-Sγ-human Cγ₁-S(ε or α)-human Cγ₂-3′
   wherein S represents a switch sequence, Cγ₁ and Cγ₂ each represent a different human constant region γ subtype G1, G2, G3 or G4 or portion thereof, and each of Sε, Sα and Sγ may be of human or non-human (e.g. murine) origin. Most preferably Sγ is Sγ3.

In a particularly preferred example of a heavy chain only animal where a human γ heavy chain constant region sequence replaces a murine γ heavy chain constant region, an animal comprising an arrangement as follows in its germline DNA can be constructed:
5′-Sγ3-human Cγ₁-Sε-human Cγ₂-3′
   wherein S represents a switch sequence, Cγ₁ and Cγ₂ represent a human constant region γ subtype G1, G2, G3 or G4 or portion thereof and may be different or the same, and each of Sε and Sγ may be of human or non-human (e.g. murine) origin. Most preferably, Sγ is Sγ3. The arrangement preferably further comprises the elements (-Sα-Cα-) oriented 3′ of Cγ₂, where Sα and Cα are of nonhuman origin or native to the nonhuman animal.

In another example of a heavy chain only animal, a human γ heavy chain constant region sequence replaces a murine γ heavy chain constant regions, an animal comprising an arrangement as follows in its germline DNA can be constructed:
5′-Sμ-Cμ-Cδ-Sγ3-human Cγ₁-Sε-human Cγ₂-3′
   wherein C represents a constant region, S represents a switch sequence, Cγ₁ and Cγ₂ each represent a human constant region γ subtype selected from the group consisting of G1, G2, G3 or G4 or portion thereof, and each of Sε, Sα and Sγ may be of human or non-human (e.g. murine) origin Most preferably Sγ is Sγ3. The arrangement preferably further comprises the elements (-Sα-Cα-) oriented 3′ of Cγ₂, where Sα and Cα are of nonhuman origin or native to the nonhuman animal. A targeting vector for use in preparing such a heavy chain only mouse can be constructed by placing a murine germline IgH locus in a suitable vector. A rearranged V_{H}DJ_{H} portion of a selected heavy chain from a lead antibody is then placed within the JH cluster and upstream of the murine μ constant region in the IgH locus. A first human heavy chain constant region of the G4 subtype replaces the murine germline DNA that encodes all of the Cγ antibody heavy chain constant regions (Cγ3, Cγ1, Cγ2b and Cγ2a) and is inserted immediately downstream of the murine germline DNA that represents Sγ3 switch sequence such that the human IgG4 region is operably linked to the murine Sγ3 switch sequence, and upstream of the Sε switch sequence (see FIG. 2). A second human heavy chain γ constant region of the G1 subtype but truncated 5′ proximal to the codon coding for the cysteine present in the hinge region and involved in the interchain disulphide bridge, representing a sequence giving rise to a Fab portion and thus in turn also to produce F(ab′)2 antibodies, replaces the murine germline DNA that encodes the Cε antibody heavy chain constant region and is inserted immediately downstream of the murine germline DNA that represents Sε switch sequence such that the human Fab-encoding heavy chain constant region is operably linked to the murine Sε switch sequence, and upstream of the murine Sα switch sequence. The targeting construct is then placed into the germline locus of the mouse ES cell by homologous recombination to obtain a heavy-chain only animal. Progeny animals obtained from a light-chain only animal and this heavy chain only animal will have B cells that produce an antibody having rearranged V_{H}DJ_{H} portion of a selected heavy chain from a lead antibody and (a) a human IgG4 constant region when challenged with LPS, or (b) a truncated IgG constant region resulting in a Fab fragment when T cells originating from a LAT Y136F mutant mouse as described in European Patent Application No. 02290610.1 are adoptively transferred to the progeny animal.

### Switches

In the development of a B lymphocyte, the cell initially produces IgM with a binding specificity determined by the productively rearranged V_{H} and V_{L} regions. Subsequently, each B cell and its progeny cells synthesize antibodies with the same L and H chain V regions, but they may switch the isotype of the H chain. The use of μ or δ constant regions is largely determined by alternate splicing, permitting IgM and IgD to be coexpressed in a single cell. The other heavy chain isotypes (γ, α, and ε) are only expressed natively after a gene rearrangement event deletes the Cμ and Cδ exons. This gene rearrangement process, isotype switching, typically occurs by recombination between so called switch segments located immediately upstream of each heavy chain gene (except δ).

The individual switch segments are between 1 and 10 kb in length, and consist primarily of short highly repetitive and G-rich sequences on the non-template strand. The repeat lengths vary from 20 to 80 nt. The upstream or donor switch region is Sμ. The downstream or acceptor switch region can be any of Sγ3, γ1, γ2b, γ2a, ε or α in mouse and any of Sγ3, γ1, α1, γ2, γ4, ε or α2 in human, in that physical order along the chromosome. The exact point of recombination differs for individual class switching and recombination crossover points can be anywhere within the switch sequences, and particularly in the case of the Sμ, upstream or downstream of the switch sequences. Dunnick et al. (1993) Nucleic Acid Res., 21, 365-372. Investigations which have used solution hybridization kinetics or Southern blotting with cDNA-derived C_{H} probes have confirmed that switching can be associated with loss of C_{H} sequences from the cell. The Sα region has been sequenced and found to consist of tandemly repeated 80-bp homology units, whereas Sγ2a, Sγ2b, and Sγ3 all contain repeated 49-bp homology units very similar to each other. (See, P. Szurek et al., J. Immunol 135:620-626 (1985) and T. Nikaido et al., J. Biol. Chem. 257:7322-7329 (1982), which are incorporated herein by reference.) All the sequenced S regions include numerous occurrences of the pentamers GAGCT and GGGGT that are the basic repeated elements of the Sμ gene (T. Nikaido et al., J. Biol. Chem. 257:7322-7329 (1982) which is incorporated herein by reference); in the other S regions these pentamers are not precisely tandemly repeated as in Sμ, but instead are embedded in larger repeat units. The Sγ1 region has an additional higher-order structure: two direct repeat sequences flank each of two clusters of 49-bp tandem repeats. (See M. R. Mowatt et al., J. Immunol. 136:2674-2683 (1986), which is incorporated herein by reference). Switch regions of human H chain genes have been found to be very similar to their mouse homologs. Switch sequences and particularly influence of switch length on recombination are reviewed in Zarrin et al, (2005) PNAS 102:2466-2470, the disclosure of which is incorporated by reference. The teachings concerning switch sequences described in Zarrin et al, and sequence lengths and segments can be used advantageously in the context of the present invention.

The targeting vectors and thus the transgenic animals according to the invention will preferably comprise a Sμ switch upstream of the Cμ coding exons, most preferably the murine Sμ switch is provided in its natural configuration upstream of the murine Cμ coding exons. The switch (S) region of the μ gene, Sμ, is located about 1 to 2 kb 5′ to the coding sequence and is composed of numerous tandem repeats of sequences of the form (GAGCT)ₙ (GGGGT), where n is usually 2 to 5 but can range as high as 17. (See T. Nikaido et al. Nature 292:845-848 (1981))

A switch recombination between μ and α genes produces a composite Sμ-Sα sequence. Typically, there is no specific site, either in Sμ or in any other S region, where the recombination always occurs. Generally, unlike the enzymatic machinery of V-J recombination, the switch machinery can apparently accommodate different alignments of the repeated homologous regions of germline S precursors and then join the sequences at different positions within the alignment. (See, T. H. Rabbits et al., Nucleic Acids Res. 9:4509-4524 (1981) and J. Ravetch et al., Proc. Natl. Acad. Sci. USA 77:6734-6738 (1980), which are incorporated herein by reference.)

Certain details of the mechanism(s) of selective activation of switching to a particular isotype may remain unknown. Although exogenous influences such as lymphokines and cytokines might upregulate isotype-specific recombinases, it is also possible that the same enzymatic machinery catalyzes switches to all isotypes and that specificity lies in targeting this machinery to specific switch regions.

The T-cell-derived lymphokines IL-4 and IFNγ have been shown to specifically promote the expression of certain isotypes: IL-4 decreases IgM, IgG2a, IgG2b, and IgG3 expression and increases IgE and IgG1 expression; while IFNγ selectively stimulates IgG2a expression and antagonizes the IL-4-induced increase in IgE and IgG1 expression (Coffman et al., J. Immunol. 136:949-954 (1986) and Snapper et al., Science 236:944-947 (1987), which are incorporated herein by reference). A combination of IL-4 and IL-5 promotes IgA expression (Coffman et al., J. Immunol. 139:3685-3690 (1987), which is incorporated herein by reference).

European Patent Application no. 02290610.1 filed Mar. 11, 2002 by Malissen, Aguado and Malissen, the disclosure of which is incorporated herein by reference, describes a mutation in the murine LAT (Linker for Activation of T cells) gene which results in impeded T cell development and an early and spontaneous accumulation of polyclonal T_{H}2 cells which chronically produce large amounts of IL-4, IL-5, IL-10 and IL-13, which in turn promotes that expression of the isotypes IgE and IgG1. In one preferred embodiment of the invention, the sequence encoding a human heavy chain constant region replaces the murine Cε in the murine germline DNA in a transgenic animal, and said animal furthermore comprises a deficiency in the LAT gene. For example the animal may comprises a LAT Y136F mutation. The human heavy chain constant region sequence can be operably linked to a murine a switch such that in a LAT Y136F animal, the animal will preferentially produce said human constant region. Alternatively, as further described herein, CD4+ T cells obtained from mice described in European Patent Application no. 02290610.1 can be provided by adoptive transfer to a transgenic mouse according to the invention in order to induce class switching to the human constant region which replaces the mouse epsilon chain, or the said CD4+ T cells can simply be incubated with cells (e.g. hybridomas) obtained from the animals of the invention in culture in order to induce class switchin.

Induction of class switching appears to be associated with sterile transcripts that initiate upstream of the switch segments (Lutzker et al., Mol. Cell. Biol. 8:1849 (1988); Stavnezer et al., Proc. Natl. Acad. Sci. USA 85:7704 (1988); Esser and Radbruch, EMBO J. 8:483 (1989); Berton et al., Proc. Natl. Acad. Sci. USA 86:2829 (1989); Rothman et al., Int. Immunol. 2:621 (1990), each of which is incorporated by reference). For example, the observed induction of the γ1 sterile transcript by IL-4 and inhibition by IFN-γ correlates with the observation that IL-4 promotes class switching to γ1 in B-cells in culture, while IFN-γ inhibits γ1 expression. Therefore, the inclusion of regulatory sequences that affect the transcription of sterile transcripts may also affect the rate of isotype switching. For example, increasing the transcription of a particular sterile transcript typically can be expected to enhance the frequency of isotype switch recombination involving adjacent switch sequences.

For these reasons, it is preferable that a construct incorporates transcriptional regulatory sequences within about 1-2 kb upstream of each switch region that is to be utilized for isotype switching. These transcriptional regulatory sequences preferably include a promoter and an enhancer element, and more preferably include the 5′ flanking (i.e., upstream) region that is naturally associated (i.e., occurs in germline configuration) with a switch region. This 5′ flanking region is typically about at least 50 nucleotides in length, preferably about at least 200 nucleotides in length, and more preferably at least 500-1000 nucleotides.

Although a 5′ flanking sequence from one switch region can be operably linked to a different switch region for preparation of a construct (e.g., the 5′ flanking sequence from the human Sγ1 switch can be grafted immediately upstream of the Sα1 switch), in some embodiments it is preferred that each switch region incorporated in the construct have the 5′ flanking region that occurs immediately upstream in the naturally occurring germ line configuration.

### Constant Regions, Modified Constant Regions.

In addition to the aforementioned constant regions and fragments and derivates thereof, it will also be possible to construct transgenic animals comprising a gene encoding a modified human heavy chain constant region. In some cases it will be preferably to use a sequence coding for a human heavy chain constant region modified (e.g. comprising one or more amino acid substitutions, insertions or deletions) to have increased binding to a human Fc receptor, particularly FcgammaR3a (CD16). The modifications will most preferably be based on an G1 or G3 human heavy chain constant region.

In another example the germline DNA of the transgenic animals comprises a human heavy chain constant region having low affinity for human Fe receptor. For example, a human heavy chain constant subtypes G4 or G2 can be used as the basis of a constant region in which the Fc portion is modified to minimize or eliminate binding to Fc receptors (see, e.g., PCT patent publication no. WO 03/101485, the disclosure of which is incorporated herein by reference). Assays, e.g., cell based assays, to assess Fc receptor binding are well known in the art. In one embodiment, a human heavy chain constant region of the G1 or G3 subtype modified to reduce binding to Fc receptors is inserted into the germline DNA of an animal. In another embodiment, a human heavy chain constant region of the G4 or G2 subtype is modified to further minimize or completely abolish binding to Fc receptors (see, e.g., Angal et al. (1993) Molecular Immunology 30:105-108, the entire disclosure of which is herein incorporated by reference.). While IgG4 isotype binds Fc receptors weakly, it has been shown that it is not totally devoid of Fc binding activity (Newman et al. (2001) Clin. Immunol. (98(2):164-174), and that an unmodified IgG4 MAb can cause cell depletion in vivo (Isaacs et al, (1996) Clin. Exp. Immunol. 106, 427). The sequence reported to be primarily responsible for the binding to Fc receptors has been defined as LLGGPS (Burton et al, (1992) Adv. Immunol. 51:1). This sequence, located at the N terminal end (EU numbering 234-239) of the heavy chain CH2 region, is conserved in human IgG1, IgG3, and murine IgG2a isotypes, all of which bind Fc receptors strongly. The wild-type sequence for the IgG4 isotype contains a phenylalanine at position 234, giving the motif FLGGPS. The murine IgG2b isotype, also a poor binder of Fc receptors, contains the sequence LEGGPS. Newman et al. (2001) incorporated the glutamic acid residue associated with murine IgG2b into the human wildtype IgG4 CH2 domain to give the sequence FEGGPS which reduced CDC and ADCC activities and virtually eliminated binding to FcRI and FcRII in vitro. In addition to the introduction of glutamic acid, the replacement of serine 228 by a proline, resulted in a molecule that was more stable than the wild-type IgG4, The IgG4 molecule tends to show inefficient formation of interchain disulfide bonds in the hinge region. The introduction of a proline was said to provide rigidity to the hinge and promote more efficient interchain bonding, and that the presence of a serine at position 228 might promotes preferential linkage of intrachain rather than inter-chain disulfide bonds by neighboring cysteine molecules. Other methods for making constant regions are known, including computing based methods such as those described in U.S. Pat. No. 6,403,312, this disclosure of which is incorporated herein by reference. Any such modifications and others can readily be made to the human heavy chain constant region to be used in the present invention.

### Additional Elements

The sequence encoding the heavy chain of the lead antibody can comprise additional elements as set forth supra.

The constructs encoding the heavy or light chain of the antibody used to construct transgenic animals of the invention can comprise additional elements. For example, cytotoxic polypeptides can be recombinantly joined to the light or heavy chain constant regions of the antibody molecule to provide an immunotherapeutic agent and included in the heavy or light chain loci.

It can be particularly advantageous to recombinantly join a detectable marker to the light or heavy chain constant region. This can then be used to produce an antibody linked to a detectable protein. The coupling of a marker to an antibody is valuable in the field of quantitative cytofluorometry and biophotonics where a very precise coupling ratio between the antibody (generally a Fab) and the fluorescent species is required. It can also be particularly advantageous to express more than one form of a given antibody. For example, it can be desirable to express an antibody in Fab form and linked to a detectable marker, and upon inducing isotype switching, expressing the same antibody in Fab form not linked to the detectable marker. In another embodiment, it would be desirable to express a given antibody or Fab fragment linked to a first marker, and upon inducing isotype switching, linked to a second marker. This can be achieved by inserting constant regions linked to a marker polypeptide and operably linked to a switch sequence. In these embodiments, the constant region used in the construct will often be of non-human origin (e.g. murine) since the antibodies are likely to be used in diagnostics or as research reagents.

Examples of detectable markers are well known. A preferred example is tandem Red, a protein obtained from a stepwise evolution of DsRed to a dimer and then either to a genetic fusion of two copies of the protein, i.e., a tandem dimer, or to a true monomer designated mRFP1 (monomeric red fluorescent protein) (Campbell et al. Proc Natl Acad Sci USA. (2002) 99(12):7877-82 and Tsien et al, U.S. Pat. No. 7,005,511 and U.S. Patent Publication no. 20060003420. Other examples include enhanced green fluorescent protein, green fluorescent protein, far-red fluorescent protein, monomeric red fluorescent protein or Renilla luciferase, Discosoma red fluorescent protein (DsRed) (Gross et al. Proc Natl Acad Sci USA. 97:11990-5 (2000).; Bevis and Glick. Nat Biotechnol. 20:83-7 (2002)), HcRed (Gurskaya et al. FEBS Lett. 507:16-20 (2001)).

Alternatively, elements, such cartilage oligomeric matrix protein, leucine zippers, such as the scZIP or scTETRAZIP constructs taught in Pack et al. (J. Mol. Biol. (1995) 246:28-34), or verotoxin subunit B (see International Patent publication no. WO 03/046560) which is hereby incorporated by reference in its entirety), can be recombinantly joined to the heavy or light chain constant regions of the antibody molecules to allow for the formation of monospecific antibody multimers or heterospecific antibodies (e.g., bispecific antibodies). Another method for preparing antibody multimers involves the joining of nucleic acid sequences encoding leucine zipper or isoleucine zipper polypeptide sequences to the heavy chain constant regions of the antibody molecules at the carboxy terminus. Examples of leucine zipper domains suitable for producing soluble multimeric proteins of the invention are those described in PCT application WO 94/10308, which is hereby incorporated by reference. Another example is a leucine zipper derived from lung surfactant protein D (SPD), as described in Hoppe et al., (1994), FEBS Letters. 344:191 and in U.S. Pat. No. 5,716,805, each which is hereby incorporated by reference in its entirety.

Other elements, such a “tags”, can be recombinantly joined to the heavy chain constant regions of the antibody molecules. Non-limiting examples of such tags are known in the art (see, for example, U.S. Pat. No. 6,342,362, hereby incorporated by reference in its entirety; Altendorf et al. [1999-WWW, 2000] “Structure and Function of the Fₒ Complex of the ATP Synthase from Escherichia coli,” J. of Experimental Biology 203:19-28, The Co. of Biologists, Ltd., G.B.; Baneyx [1999] “Recombinant Protein. Expression in Escherichia coli,” Biotechnology 10:411-21, Elsevier Science Ltd.; Eihauer et al. [2001] “The FLAG™ Peptide, a Versatile Fusion Tag for the Purification of Recombinant Proteins,” J. Biochem Biophys Methods 49:455-65; Jones et al. [1995] J. Chromatography 707:3-22; Jones et al. [1995] “Current Trends in Molecular Recognition and Bioseparation,” J. of Chromatography A. 707:3-22, Elsevier Science B. V.; Margolin [2000] “Green Fluorescent Protein as a Reporter for Macromolecular Localization in Bacterial Cells,” Methods 20:62-72, Academic Press; Puig et al. [2001] “The Tandem Affinity Purification (TAP) Method: A General Procedure of Protein Complex Purification,” Methods 24:218-29, Academic Press; Sassenfeld [1990] “Engineering Proteins for Purification,” TibTech 8:88-93; Sheibani [1999] “Prokaryotic Gene Fusion Expression Systems and Their Use in Structural and Functional Studies of Proteins,” Prep. Biochem. & Biotechnol. 29(1):77-90, Marcel Dekker, Inc.; Skerra et al. [1999] “Applications of a Peptide Ligand for Streptavidin: the Strep-tag”, Biomolecular Engineering 16:79-86, Elsevier Science, B. V.; Smith [1998] “Cookbook for Eukaryotic Protein Expression: Yeast, Insect, and Plant Expression Systems,” The Scientist 12(22):20; Smyth et al. [2000] “Eukaryotic Expression and Purification of Recombinant Extracellular Matrix Proteins Carrying the Strep H Tag”, Methods in Molecular Biology, 139:49-57; Unger [1997] “Show Me the Money: Prokaryotic Expression Vectors and Purification Systems,” The Scientist 11(17):20, each of which is hereby incorporated by reference in their entireties), or commercially available tags from vendors such as such as Amersham Biosciences Corp., (Piscataway, N.J.), STRATAGENE (La Jolla, Calif.), NOVAGEN (Madison, Wis.), QIAGEN, Inc., (Valencia, Calif.), Or InVitrogen (San Diego, Calif.)). Other “tags” or handles suitable for attachment to the heavy chain constant regions of the antibody molecules are provided in Table 3.

In certain embodiments, the tag(s) can be a polyhistidine tag selected from the group consisting of (His)ₙ where n is an integer from 3, 4, 5, 6, 7, 8, 9, 10, 11, 12, 13, 14, 15, 16, 17, 18, 19 or 20 or more (alternatively, n is an integer of at least 3). In some embodiments n is 5 or 6. Another polyhistidine tag that can be used is [His-(Xaa)]ₙ where n is an integer from 3, 4, 5, 6, 7, 8, 9, 10, 11, 12, 13, 14, 15, 16, 17, 18, 19 or 20 or more (alternatively, n is an integer of at least 3) and wherein Xaa can be any amino acid. In some embodiments n is 5 or 6. Yet another polyhistidine tag [(Xaa)₂-His]₄-Xaa-His-Xaa-His-(Xaa)₂]; wherein Xaa can be any amino acid. One exemplary [His-(Xaa)]₆ affinity tag can be His-Asn-His-Asn-His-Asn-His-Asn-His-Asn-His-Asn. An exemplary [(Xaa)₂-His]₄-Xaa-His-Xaa-His-(Xaa)₂] affinity tag can be Lys-Asp-His-Leu-Ile-His-Asn-Val-His-Lys-Glu-His-Ala-His-Asn-Lys.

In other embodiments, the tag(s) can be Glutathione S-transferase (GST). Plasmids for the expression of fusion proteins containing GST are commercially available from Amersham Biosciences Corp. (Piscataway, N.J.). Non-limiting examples of such plasmids are the family of pGEX vectors sold by Amersham. Alternatively, nucleic acids encoding GST can be inserted into the constructs of the subject invention.

Another tag suitable for use in the subject invention is the c-myc tag. The c-myc epitope tag has the sequence AEEQKLISEEDLL. Insertion of this sequence into recombinant antibodies of the subject invention can allow for their purification using known affinity chromatography techniques and antibodies specific for the c-myc epitope tag. Kits that facilitate such purification are available from any number of commercial vendors as indicated supra.

### Exemplary Heavy Chain Only Animal Targeting Construct for Expression of Detectable Antibodies

In one example of a heavy chain only animal, where a murine γ heavy chain constant region recombinantly joined to a linker and a fluorescent protein (EGFP in this example) sequence replaces a murine γ heavy chain constant region, an animal comprising an arrangement as follows in its germline DNA can be constructed:
5′-Sμ-Cμ-Cδ-Sγ3-murine Cγ₁-Sε-murine Cγ₂-linker-EGFP-3′
   wherein C represents a constant region, S represents a switch sequence, Cγ₁ and Cγ₂ each represent a murine constant region γ subtype selected from the group consisting of G1, G2, G3 or G4 or portion thereof, and each of Sε, Sα and Sγ are preferably of murine origin. Most preferably Sγ is Sγ3. The arrangement preferably further comprises the elements (-Sα-Cα-) oriented 3′ of Cγ₂, where Sα and Cα are of nonhuman origin or native to the nonhuman animal. A targeting vector for use in preparing such a heavy chain only mouse can be constructed by placing a murine germline IgH locus in a suitable vector. A rearranged V_{H}DJ_{H} portion of a selected heavy chain from a lead antibody (e.g. the KT3 mAb, a rat antibody specific for the mouse CD3 epsilon subunit of the TCR complex) is then placed within the JH cluster and upstream of the murine μ constant region in the IgH locus. A first murine heavy chain constant region of the G1 subtype but truncated 5′ proximal to the codon coding for the cysteine present in the hinge region and involved in the interchain disulphide bridge, representing a sequence giving rise to a Fab portion and thus in turn also to produce F(ab′)2 antibodies, replaces the murine germline DNA that encodes all of the Cγ antibody heavy chain constant regions (Cγ3, Cγ1, Cγ2b and Cγ2a) and is inserted immediately downstream of the murine germline DNA that represents Sγ3 switch sequence such that the human IgG1 region is operably linked to the murine Sγ3 switch sequence, and upstream of the Sε switch sequence. A second murine heavy chain γ constant region of the G1 subtype and also truncated 5′ proximal to the codon coding for the cysteine present in the hinge region and involved in the interchain disulphide bridge, representing a sequence giving rise to a Fab portion and thus in turn also to produce F(ab′)2 antibodies, and recombinantly joined to a linker and EGFP replaces the murine germline DNA that encodes the Cε antibody heavy chain constant region and is inserted immediately downstream of the murine germline DNA that represents Sε switch sequence such that the human Fab-encoding heavy chain constant region is operably linked to the murine Sε switch sequence, and upstream of the murine Sα switch sequence. The targeting construct is then placed into the germline locus of the mouse ES cell by homologous recombination to obtain a heavy-chain only animal. Progeny animals obtained from a light-chain only animal and this heavy chain only animal will have B cells that produce an antibody having rearranged V_{H}DJ_{H} portion of the heavy chain from the KT3 antibody and (a) a truncated IgG constant region resulting in a Fab fragment when challenged with LPS, or (b) a truncated IgG constant region resulting in a Fab fragment and linked to a EGFP protein when T cells originating from a LAT Y136F mutant mouse as described in European Patent Application No. 02290610.1 are adoptively transferred to the progeny animal or incubated in wells together with cells derived from the progeny animal.

### Targeting Vectors

The targeting vectors of the invention comprise recombinant DNA vectors including, but not limited to, plasmids, phages, phagemids, cosmids, viruses and the like which contain the sequences to be inserted into the germ-line DNA of a non-human animal.

While any suitable method can be used to construct the “light chain only animals” and “heavy chain only animals” of the invention, a simple and convenient method relies on the use of targeting vectors that permit efficient vector construction and targeted insertion into the a nonhuman animal cell's germline DNA based on homologous recombination. The “light chain only animals” and “heavy chain only animals” can be conveniently constructed with the use of a targeting vectors that comprise (as a starting point) all or a portion of the an IgH locus (of human or nonhuman origin), and are modified using the elements as described herein.

The most convenient means for preparing the cells and transgenic animals according to the invention is to use targeting vectors designed to be incorporated by homologous recombination. Cultured mammalian cells will integrate exogenous plasmid DNA into chromosomal DNA at the chromosome location which contains sequences homologous to the plasmid sequences. (Folger, et al. 1982, Mol. Cell. Biol. 2, 1372-1387; Folger, et al., 1984, Symp. Quant. Biol. 49, 123-138; Kucherlapati, et al., 1984, Proc. Natl. Acad. Sci. USA 81, 3153-3157; Lin, et al., 1985, Proc. Natl. Acad. Sci. USA 82, 1391-1395; Robert de Saint Vincent, et al., 1983, Proc. Natl. Acad. Sci. USA 80, 2002-2006; Shaul, et al., 1985, Proc. Natl. Acad. Sci. USA 82, 3781-3784). Mammalian cells also contain the enzymatic machinery to integrate plasmid DNA at random chromosomal sites, referred to as nonhomologous recombinations. The frequency of homologous recombination has been reported to be as high as between 1/100 to 1/1000 of the recombinational events, while the majority of recombinations result from nonhomologous interactions (Thomas et al., 1986, Cell 44:419-428; Smithies et al., 1985, Nature 317:230-234; Shaul, et al., 1985, Proc. Natl. Acad. Sci. USA 82, 3781-3784; Smith, et al., 1984, Symp. Quant. Biol. 49, 171-181; Subramani, et al., 1983 Mol. Cell. Biol. 3, 1040-1052). The existence of the cell machinery for homologous recombination makes it possible to modify endogenous genes in situ. In some instances, conditions have been found where the chromosomal sequence can be modified by introducing into the cell a plasmid DNA which contains a segment of DNA homologous to the target locus and a segment of new sequences with the desired modification (Thomas et al., 1986, Cell 44:419-428; Smithies et al., 1985, Nature 317:230-234; Smith, et al., 1984, Symp. Quant. Biol. 49, 171-181). Homologous recombination between the mammalian cell chromosomal DNA and the exogenous plasmid DNA can result in the integration of the plasmid or in the replacement of some of the chromosomal sequences with homologous plasmid sequences. The process of replacing homologous DNA sequences is referred to as gene conversion. Both the integration and the conversion events can result in positioning the desired new sequence at the endogenous target locus.

Generally a single targeting vector is used containing all elements to be inserted in the host genome is used. The vector will usually include the rearranged V_{H}DJ_{H} or VJ gene and/or at least one human constant region gene, and regions of homology to the host target, i.e. the region of the chromosome that will be replaced with the human sequence. The homologous region will usually be at least about 20, 30, 50 or 100 bp, in some cases at least about 1 kb, but usually not more than about 10 kb in length. If a non-mammalian recombinase, e.g. Cre, Flip, etc., is to be used, the homologous region will contain the entire region to be replaced, having recombinase recognition sites, e.g. loxP, frt, flanking the selectable marker and homologous region. Optionally, as further described in the herein the vector contains additional elements, including switch sequences and one or more constant region genes from the host species or from humans (e.g. human or murine μ and δ constant regions).

The target sequence (for homologous recombination with the host) and the construct to be inserted into the host DNA are positioned in the targeting vector so that transfection of the appropriate cell line (e.g. and ES cell) with the targeting vector results in targeted homologous recombination and site specific insertion of the replacement gene into the host germline DNA. The targeting vectors of the invention may contain additional genes which encode selectable markers including but not limited to enzymes which confer drug resistance to assist in the screening and selection of transfectants; alternatively the vectors of the invention may be cotransfected with such markers. Other sequences which may enhance the occurrence of recombinational events may be included as well. Such genes may include but are not limited to either eucaryotic or procaryotic recombination enzymes such as REC A, topoisomerase, REC 1 or other DNA sequences which enhance recombination such as CHL Furthermore, sequences which enhance transcription of chimeric genes produced by homologous recombination may also be included in the vectors of the invention; such sequences include, but are not limited to, inducible elements such as the metallothionine promoter. Various proteins, such as those encoded by the aforementioned genes may also be transfected in order to increase recombination frequencies.

Systems for Efficient Targeting Vector Construction in E. coli

Several systems useful in the methods of the invention permit rapid and efficient construction of targeting vectors that can thereafter be used for insertion into a genome. One example is the Red/ET recombination system (Zhang, Y., Buchholz, F., Muyrers, J. P. P. and Stewart, A. F. (1998). Nature Genetics, 20, 123-128; and Muyrers, J. P. P., Zhang, Y. and Stewart A. F. (2001). Trends in Biochemical Sciences, 26, 325-31). In Red/ET recombination, also referred to as lambda-mediated recombination, target DNA molecules are precisely altered by homologous recombination in strains of E. coli which express phage-derived protein pairs, either RecE/RecT from the Rac prophage, or Reda/Redb from lambda phage. These protein pairs are functionally and operationally equivalent. RecE and Reda are exonucleases, and RecT and Redb are DNA annealing proteins.

Another example is the “Recombineering” system (available from NCI Frederick, Frederick, Md.), a method based on homologous recombination in E. Coli using recombination proteins provided from λ phage. The targeting vector is constructed in bacterial strains containing a defective λ prophage inserted into the bacterial genome. The phage genes of interest, exo, bet, and gam, are transcribed from the λPL promoter. This promoter is repressed by the temperature-sensitive repressor cI857 at 32° C. and derepressed (the repressor is inactive) at 42° C. After a 15 minute heat-shock at 42° C. a sufficient amount of recombination proteins are produced. exo is a 5′-3′ exonuclease that creates single-stranded overhangs on introduced linear DNA. bet protects these overhangs and assists in the subsequent recombination process. gam prevents degradation of linear DNA by inhibiting E. Coli RecBCD protein. Linear DNA (PCR product, oligo, etc.) with sufficient homology in the 5′ and 3′ ends to a target DNA molecule already present in the bacteria (plasmid, BAC, or the bacterial genome itself) can be introduced into heat-shocked and electrocompetent bacteria using electroporation. The introduced DNA will now be modified by exo and bet and undergo homologous recombination with the target molecule. Protocols are provided at http://recombineering.ncifcrf.gov.

Various markers may be employed for selection. These markers include the HPRT minigene (Reid et al. (1990) Proc. Natl. Acad. Sci. USA 87:4299-4303), the neo gene for resistance to G418, the HSV thymidine kinase (tk) gene for sensitivity to gancyclovir, the hygromycin resistance gene, etc. The recombination vehicle may also contain viral recognition sequences, e.g. SV40, etc., additional sequences to amplify gene expression and the like.

Once prepared, the construct(s) is inserted into a host cell's germline DNA by transforming a host cell with the targeting vector(s). Preferably the host cell is an embryonic stem (ES) cell. After transfection, the embryonic stem cells are grown in culture under conditions that select for cells expressing the selectable marker gene. Those cells are then screened to determine whether the recombination event took place at the homologous chromosome region. Such screening may be performed by any convenient method, including Southern blotting for detection of differentially sized fragments, PCR amplification, hybridization, etc.

Cells having the desired recombination are injected into blastocysts of the host mammal. Blastocysts may be obtained from females by flushing the uterus 3-5 days after ovulation. At least one, and up to thirty, modified embryonic stem cells may be injected into the blastocoel of the blastocyst. After injection, at least one and not more then about fifteen of the blastocysts are returned to each uterine horn of pseudo-pregnant females. Females are then allowed to go to term, and the resulting litter is screened for mutant cells having the construct. In this manner, light chain only and heavy chain only animals are obtained.

Subsequent breeding allows for germ line transmission of the altered locus. One can choose to breed heterozygous offspring and select for homozygous offspring, (i.e. those having the human gene segment present on both chromosomes) from the heterozygous parents, or the embryonic stem cell may be used for additional homologous recombination.

### Progeny Animals and use Thereof

The subject invention further provides “progeny animals” arising from the mating of “light chain only” and “heavy chain only” (or HCOA2 or HCOA3) animals and it is preferred that the animals used in the mating process contain antibody heavy and light chains derived from the same human, humanized or chimeric antibody molecule.

Progeny animals arising from the mating step can be, subsequently, immunized with antigen specific for the human, humanized or chimeric antibody to induce the clonal expansion of B-cells.

For animals retaining the ability to undergo hypermutation of the V_{H}DJ_{H} segments, immunization with antigen specific for the human, humanized or chimeric antibody will also be useful to induce somatic hypermutation of the V_{H}DJ_{H} segment.

Where the mating of a light chain only and heavy chain only animal, preferably a HCOA2 or HCOA3 animal has been performed, the progeny animals can be treated to induce a class switch of the antibody produced by the B-cells to the desired isotype(s). This can be carried out using any suitable method; in one example, a cytokine is administered to the progeny animal. In another example, LPS is administered to the progeny animal to stimulate a class switch of the antibody produced by the B-cells from IgM to IgG₄ (in addition to immunization with specific antigen; see, for example, FIG. 3). In another example, cells obtained from a mutant mouse as described in European Patent Application no. 02290610.1 are adoptively transferred to the progeny animal. In another embodiment, no particular treatment of the progeny animal is required to induce switching to a desired isotype; for example if an animal harbors a LAT Y136F mutation as described in European Patent Application no. 02290610.1, the animal will preferentially produce antibodies from the IgE and IgG1 subtypes (or the human heavy chain constant region subtype replacing the murine counterpart).

In the example of FIG. 2, a human heavy chain constant region G1, G2, G3 or G4 is incorporated upstream of the Sα switch sequence and downstream of the Sγ3 switch sequence, replacing the murine germline DNA that encodes the γ and ε heavy chain constant regions, and a human heavy chain γ constant region of the G1 subtype but truncated 5′ proximal to the codon coding for the cysteine present in the hinge region and involved in the interchain disulphide bridge replaces the murine germline DNA that encodes the Cε antibody heavy chain constant region and is inserted immediately downstream of the murine germ line DNA that represents Sε switch sequence and upstream of the murine Sα switch sequence. A B cell from this animal will produce (a) an antibody Fab fragment by (i) default if the progeny animal harbors a LAT Y136F mutation or (ii) upon adoptive transfer of T cells from an animal harboring a LAT Y136F mutation, and (b) a full antibody (for example of the G1 or G4 subtype) upon administration of LPS. When somatic hypermutation is induced with antigen, inducing is preferably carried out following immunization with antigen.

As mentioned, any suitable class switching step can be used. In exemplary HCOA3 animals class switching can be induced by LPS to induce the expression of the human heavy chain that replaces the mouse Cγ3, Cγ1, Cγ2b and Cγ2a region set or α heavy chain constant region. Class switching can be induced to induce the expression of the human heavy chain replacing the mouses ε chain, by the treatment of the progeny animal of the invention with CD4 T cells derived from mouse described in European Patent Application no. 02290610.1.

Accordingly, the methods of the subject invention, generally, comprises the construction of: 1) a first non-human animal comprising a sequence encoding at least a rearranged V region of a heavy chain of a human, chimeric or humanized lead antibody operably linked to germline or modified constant region sequences; and 2) a second non-human animal comprising a sequence encoding at least the rearranged variable region of a light chain of a particular human, chimeric or humanized lead antibody operably linked to germline or modified constant region sequences.

These animals are then mated and the offspring/progeny tested for the production of antibodies capable of specifically binding to the antigen to which the human, chimeric or humanized antibody is specific. If desired, the progeny having the desired phenotype (e.g., producing antibodies of a desired binding specificity) are challenged with specific antigen and/or LPS or other treatment to stimulate the clonal expansion of the B-cells producing the human, chimeric or humanized antibody and/or induce somatic hypermutation of the V_{H}DJ_{H} and VLJL segments and thus the affinity maturation of the known monoclonal, and/or cause a class switch from IgM production to the production of IgG antibodies of a desired subtype. A particular advantageous aspect of the invention is that the animal—preferably a mouse—will produce a substantially monoclonal population of B cells producing the mAb of interest. Fusion should result in a large number of clones displaying the same or very similar mAb, and corresponding hybridoma can be selected to retain only the best producers as assessed by known methods. The invention thereby provides methods for obtaining, identifying or producing cells, preferably B cells and hybridomas, capable of increased levels of production of an antibody of interest.

The present invention therefore provides a method for increasing the affinity of an antibody for its specific antigen comprising inducing the somatic hypermutation of a lead antibody-derived sequence or lead sequence in vivo. In this aspect of the invention, animals are immunized (e.g., repeatedly immunized—e.g. at least five to twenty times) with specific antigen and the B-cell clones of the animal repeatedly expanded and selected in response to the antigen. The animal of the present invention therefore permit the preparation of an affinity matured antibody. An “affinity matured” antibody is one with one or more alterations in one or more CDRs thereof which result an improvement in the affinity, of the antibody for antigen, compared to a parent antibody which has not been altered. Preferred affinity matured antibodies will have nanomolar or even picomolar affinities for the target antigen. Preferably, the method comprises improving affinity by an antibody for a target antigen by at least 20%, 30%, 50%, 75%, 90%, 100%, 200% or 1000%, or at least 1, 2, 3 or 4-log, over the lead antibody.

In preferred embodiments, the method includes a step of selecting or isolating B-cells from the progeny animals producing a human chimeric or humanized antibody of interest. As discussed, the invention provides a method of preparing a hybridoma producing a human chimeric or humanized antibody of interest, methods of obtaining B cells and derivatives or progeny thereof (e.g. fused cells such as a hybridoma) having improved production of a human, chimeric or humanized antibody, and methods of obtaining improved antibodies (e.g. affinity matured antibodies). Accordingly, the B cells can be selected based on the appropriate characteristics such as simply positive for antibody production, or antibody production characteristics (e.g. level or amount or any other criteria), the nature of the antibody produced (affinity, subtype, specificity, etc.). In one embodiment, the invention encompasses an isolated hybridoma expressing a human, chimeric or humanized antibody. The present invention also concerns a method for producing a human chimeric or humanized antibody of interest using a progeny animals, a B cell or a hybridoma of the present invention.

Preferably, B cells obtained from an animal are fused to myeloma cells to produce hybridomas (immortalized cell lines). Advantageously, hybridomas as selected for their ability for high level (quantity) production of the human, chimeric or humanized antibodies. Exemplary myeloma cells suitable for use in the production of monoclonal antibodies using B-cells derived from certain mammals are set forth in Table 2.

After antibodies of a desired specificity have been identified in a progeny animal and, optionally immortalized via fusion with myeloma cells. These cells can be used to produce antibodies in desired quantities, and antibodies produced by such cells can be isolated and used for any desired application, e.g. therapeutic, diagnostic, research.

The invention also provides a method for identifying candidate hybridomas which secrete a monoclonal antibody of the subject invention. In this aspect of the invention, the supernatant(s) of individual or pooled hybridoma clones is contacted or incubated with a predetermined antigen, typically an antigen which is immobilized by adsorption onto a solid substrate (e.g., a microtiter well), under binding conditions to select antibodies having the predetermined antigen binding specificity. An antibody that specifically binds to huuman constant regions is also contacted or incubated with the hybridoma supernatant and predetermined antigen under binding conditions so that the antibody selectively binds to at least one human constant region epitope but substantially does not bind to murine constant region epitopes; thus forming complexes consisting essentially of hybridoma supernatant (transgenic monoclonal antibody) bound to a predetermined antigen and to an antibody that specifically binds human constant regions (and which may be labeled with a detectable label or reporter). Detection of the formation of such complexes indicates hybridoma clones or pools which express a human immunoglobulin chain.

In one embodiment the candidate hybridomas are first screened for the ability to produce antibodies that bind specific antigen. Thus, according to the method, a transgenic animal of the invention is immunized with the predetermined antigen to induce an immune response. B cells are collected from the animal and fused to appropriate myeloma cells to produce hybridomas. The hybridomas are then screened for specific binding to an antigen and then for the isotype of antibody. Screening can be carried out using standard techniques as described in, e.g., Harlow and Lane, Antibodies: A Laboratory Manual, Cold Spring Harbor, N.Y. (1988).

### Further Modification to Antibodies

If desired, the antibodies produced by the B cells can be modified in any suitable process. For example, the binding affinity of the antibodies can be increased via various methods known in the art. For example, binding characteristics can be improved by direct mutation, methods of affinity maturation, phage display, or chain shuffling within the nucleic acids encoding the antibody molecules. For example, individual residues or combinations of residues can be randomized so that in a population of otherwise identical antigen binding sites, all twenty amino acids are found at particular positions. Binding characteristics can also be improved by methods of affinity maturation. (See, e.g., Yang et al. (1995) J. Mol. Bio. 254, 392-403; Hawkins et al. (1992) J. Mol. Bio. 226, 889-896; or Low et al. (1996) J. Mol. Bio. 250, 359-368 (each of which is hereby incorporated by reference in its entirety, particularly with respect to methods of increasing the binding affinity of antibodies)). Methods known in the art include for example, Marks et al. Bio/Technology, 10:779-783 (1992) describes affinity maturation by VH and VL domain shuffling; random mutagenesis of CDR and/or framework residues is described by: Barbas et al. Proc Nat. Acad. Sci, USA 91:3809-3813 (1994); Schier et al. Gene, 169:147-155 (1995); Yelton et al. J. Immunol., 155:1994-2004 (1995); Jackson et al., J. Immunol., 154(7):3310-9 (1995); and Hawkins et al, J. Mol. Biol., 226:889-896 (1992).

Strategies for antibody optimization are sometimes carried out using random mutagenesis. In these cases positions are chosen randomly, or amino acid changes are made using simplistic rules. For example all residues may be mutated to alanine, referred to as alanine scanning. WO 9523813 (which is hereby incorporated by reference in its entirety) teaches in vitro methods of increasing antibody affinities utilizing alanine scanning mutagenesis. Alanine scanning mutagenesis cans also be used, for example, to map the antigen binding residues of an antibody (Kelley et al., 1993, Biochemistry 32:6828-6835; Vajdos et al., 2002, J. Mol. Biol. 320:415-428). Sequence-based methods of affinity maturation (see, U.S. Pat. Application No. 2003/022240 A1 and U.S. Pat. No. 2002/177170A1, both hereby incorporated by reference in their entireties) may also be used to increase the binding affinities of antibodies.

Further aspects and advantages of this invention are disclosed in the following experimental section, which should be regarded as illustrative and not limiting the scope of this application.

### EXAMPLES

### Example 1

### Engineering of the Mouse Ig H Locus

Two mouse BACs denoted RP23-351J19 and RP23-109B20, and corresponding to the mouse IgH locus were selected from a BAC library (Osoegawa K et al. (2000) Genome Res. 10:116-128, the disclosure of which is incorporated herein by reference in its entirety). They show a 76 kb overlap and each covers part of the region containing the diversity (D), and junction (J) gene segments, and the constant (C; IgG3 to IgA) genes (FIG. 5A). The integrity of the sequences harbored by the two BACs was determined using pulsed-field gel electrophoresis.

### Fusing BAC RP23-351J19 to BAC RP23-109B20.

In a first step, the two BACs are fused to generate a recombinant BAC containing the D and J gene segments as well as the C genes. Two strategies are carried out.

### Strategy 1.

First, a puromycin resistance cassette (de la Luna S et al, (1992) Methods Enzymol. 216:376-85, the disclosure of which is incorporated herein by reference) (“Puro”) is introduced into BAC RP23-109B20. This cassette is synthesized using oligonucleotide primers corresponding (1) to sequence located at the 3′ end of the IgH cluster and to sequences located at the extremity of BAC RP23-109B20 contiguous to the T7 sequence. As shown in FIG. 5B, one of the oligonucleotide primer contains a I-Sce I restriction site (to facilitate the linearization of the final recombination substrate, see below). Targeting of the synthesized puromycin cassette into BAC RP23-109B20 results in the deletion (“shaving”) of 63 kb of sequences encompassing the whole D gene segment cluster. This intermediate product called RP23-10920puro is grown and digested with SnaBI Digesting RP23-109B2Opuro with Sna BI disables the vector used to construct the BAC library. This strain bacteria is also transfected with the plasmid pSC101-BAD-gbaA (coding for the ET recombinase, Stewart, A. F., Zhang, Y., and Buchholz, F. 1997. Novel DNA cloning method. European Patent Application No. 98 963 541.2 (or PCT/EP98/07945). Bacteria growing in the presence of both chloramphenicol and puromycin thus contain a recombinant BAC (denoted RP23-351J19puro) that displays the structure shown in FIG. 5D. The expected structure is verified by field-pulse gel electrophoresis and partial sequencing.

### Strategy 2.

A backup strategy 2 can be used as an alternative to strategy 1 above. A blasticidine (“Blast”) resistance cassette (Itaya M et al, J Biochem (1990) 107:799-801) is introduced into BAC RP23-351J19 using homologous sequences flanking the 3′ end of the IgA C gene (FIG. 5C). The resulting BAC is denoted RP23-351J19blast. Microgram amounts of BAC RP23-351J19 blast and BAC RP23-109B20puro (see 1.1.1) are prepared. BAC RP23-351J19blast is digested with MluI and BsiWI, whereas BAC RP23-109B20puro is restricted by Mlu I and BsiWI. The MluI-BsiWI fragment encompassing the IgG3C, IgDC and IgMC genes as well as the JH gene cluster are cloned into the MluI-BsiWI restricted BAC RP23-351J19 blast to give rise to BAC RP23-351J19puro/blast (FIG. 5D).

### Substitution of the Sequences Coding for the Mouse IgG2b, IgG1, IgG3c and IgG2a C Genes by the Sequence Coding for the Human IgG1 C Gene.

### Step 1

This substitution is carried out by recombinogenic engineering using either BAC RP23-351J19puro or BAC RP23-351J19puro-blast. The IgA and IgE C genes located at the 3′ end of the IgCH cluster are first deleted by homologous recombination using an Ampicillin-based cassette flanked by homology arms corresponding to sequences located at the 5′ and of the IgE C gene and to sequences located at the 3′-most end of the IgH C cluster. In the case BAC RP23-351J19 puro-blast is used, this step is also used to remove the blasticidine cassette. Note that this approach specifies the extent of the 5′ homology arm.

### Step 2. Construction and Insertion of a Human IgG1-Lox 511-Hygro-lox 511 Cassette

A 3.2 kb fragment straddling exons CH1, H, CH2 and CH3 of the human IgG1 C gene are synthesized by PCR using BAC RP11-417P24 (Osoegawa K et al, (2001) Genome Res. 11:483-96) as a template, and a 5′ end primer with sequence complementary to the beginning of the human IgG1 CH1 exon (primer a), and a 3′-end primer complementary to the 3′end of the human IgG1 CH3 exon (primer b). Sequences complementary to the splicing site located to the 5′ end of the CH1 exon of the mouse IgG3 C gene are abutted to the 5′ end of primer a. Sequences complementary to the intron flanking the 3′ end of the CH3 exon of the mouse IgG2a C gene are abutted to the 3′ end of primer b.

The corresponding PCR product is sequenced and cloned. A lox511-flanked hygromycin resistance cassette (Giordano T et al, (1990) Gene 88:285-288, the disclosure of which is incorporated herein by reference) is inserted at the 3′ end of the CH3 exon of the IgG2bc gene (FIG. 5D). The resulting human IgG1-lox511-Hygro-lox511 cassette is inserted into BAC RP23-351J19 puro or BACRP23-351J19 puro/blast through recombineering.

### Construction and Insertion of a VHDHJH^{16D10} lox P-Tace Neo-lox P Cassette

The VH gene used by hybridoma “IPH1” was identified and denoted VHDHJH^{IPH1}. This hybridoma secretes an IgM equipped with a kappa light chain. A genomic fragment encompassing the promoter of the VHDHJH^{IPH1} gene and ending up at the 3′ end of the JH gene segment used by the VHDHJH^{IPH1} gene is synthesized by PCR from DNA extracted from the IPH1 hybridoma. The primer located at the 5′ end of the VHDHJH promoter incorporates a sequence homologous to sequences flanking the 5′ end of the JH gene cluster. A lox P-flanked Cre-neo auto-deleter cassette (Tace-Neo cassette; Bunting M et al (1999) Genes Dev. 13:1524-8, the disclosure of which is incorporated herein by reference) is inserted in the 3′ end of the VHDHJH^{IPH1} fragment as shown in FIG. 5D. The VHDHJH^{IPH1} lox P-Tace Neo-LoxP cassette is inserted into BAC RP23-351J19 puro or BAC RP23-351J19 puro blast by recombinogenic engineering as shown in FIG. 5D. 5′ and 3′ single-copy probes and appropriate restriction sites are defined to ensure that homologous recombination had occurred in ES cells at each end of the intended insertion.

### Isolation of Recombinant ES Clone.

BAC DNA are prepared using five-liter culture and purified on Cesium Chloride gradient. After digestion with I-Sce I, the targeting construct is extracted with phenol-chloroform, precipitated with ethanol, and resuspended in PBS.

Bruce 4 ES cells are electroporated with the I-Sce I linearized BAC VHDHJH^{IPH1}-mCM-mCD-hCG1. 24 hr after electroporation, drug selection is started at the following concentrations: G418: 200 μg/ml and hygromycin (160 mg/ml). Selection in G418 and hygromycin, colonies are screened for homologous recombination by Southern blot analysis.

### Production of Mutant Mice.

Mutant ES are injected into Balb/c blastocysts. The hygromycin and neomycin cassette are self-excised during male germline transmission. The result of the knock-in approach is a “rearranged” mouse IgH locus containing a VHDHJH^{IPH1} gene driven by its own promoter, a loxP site, the mouse CM and CD genes, the human CG1 and a Lox511 site.

### Engineering of the Mouse Ig C Kappa Locus.

The mouse Ig C kappa locus presents a rather simple organization when compared to the mouse I_{g}H locus. Owing to this attribute, and as outlined in FIGS. 5E and 5F, only three recombineering steps are required to obtain the proper recombination substrate.

### Subcloning of the JK Gene Cluster and CK Gene.

The JK gene cluster and CK gene are subcloned into pUC by recombineering using BAC RP23-435I4 as the starting template (Osoegawa K et al, (2000) Genome Res 10:116-128). The resulting subclone will be denoted “JK cluster-CK gene”.

As shown in FIG. 5F, a genomic fragment corresponding to the promoter of the VKJK^{IPH1} gene and to the VKJK^{IPH1} gene itself are isolated from hybridoma IPH1. A lox P-flanked self-deleting neo resistance cassette is inserted at the 3′ end of the VKJK^{IPH1} gene and a region homologous to sequences flanking the 5′ end of the JK cluster abutted to the 5′ end of the VKJK^{IPH1} promoter. This fragment is introduced by recombineering into the “JK cluster-CK gene” subclone as shown in FIG. 5F.

The mouse CK gene is then replaced by the human CK gene using a strategy identical to the one described for the introduction of the human IgG1 C gene into the mouse IgH locus using the RP11-601N4 (see step above “Construction and insertion of a VHDHJH^{IPH1} lox P-Tace Neo-lox P cassette” and FIG. 5F; Osoegawa K et al, (2001) Genome Res.11:483-96).

Isolation of recombinant ES clones and production of mutant mice with a humanized CK locus and a “rearranged” VKJK^{IPH1} gene is conducted as described for the IgH locus.

### Example 2

### Engineering of a Transgenic Animal Expressing an Antibody Linked to a Marker

A transgenic mouse is generated where one C gene of the IgH locus (preferentially the E or G1 isotype of the C domain, to benefit of the possibility' to control their expression using LatY136F inducer T cells via isotype switching) are replaced by a sequence composed of a cDNA coding for a linker-EGFP or linker-tandem Red sequence.

To prove the feasability of the approach, a construct is made in a first step to test the expression of the antibody expressed as a single open reading fram a Fab-linker-EGFP version of the KT3 mAb (a rat antibody specific for the mouse CD3 epsilon subunit of the TCR complex).

Accordingly, we have expressed in the X63-AgX653, a cassette containing as a single open reading frame a sequence corresponding to:
a. the leader of the KT3 VH gene,
b. the KT3 VH gene,
c. the KT3 CH1 (IgG2a) sequence,
d. a <<D2AK2>> linker,
e. a monomeric form of EGFP, a furin/24 cleavage site,
f. the complete KT3 kappa light chain, and
g. a splice site to facilitate the expression in an ad hoc expression vector.

A schematic of the construction is shown in FIG. 6.

Following testing of the construct, the transgenic animals which express the antibody according to the methods of the invention can be generated. In brief, animals are generated as in Example 1.

In order to generate a progeny animal expressing antibodies linked to a detectable protein, and the same antibody without detectable marker, a murine γ heavy chain constant region sequence replaces a first murine γ heavy chain constant region, and a murine γ heavy chain constant region recombinantly joined to a linker and a fluorescent protein (EGFP in this example) sequence replaces a second murine γ heavy chain constant region. The animal has an arrangement as follows in its germline DNA:
5′-Sμ-Cμ-Cδ-Sγ3-murine Cγ₁-Sε-(murine Cγ₂-linker-EGFP)-3′
   wherein C represents a constant region, S represents a switch sequence, Cγ₁ and Cγ₂ each represent a murine constant region G1 subtype and also truncated 5′ proximal to the codon coding for the cysteine present in the hinge region and involved in the interchain disulphide bridge, representing a sequence giving rise to a Fab portion, and each of Sε, Sα and Sγ are of murine origin. The arrangement further comprises the elements (-Sα-Cα-) oriented 3′ of Cγ₂, where Sα and Cα are of murine origin. A targeting vector for use in preparing such a heavy chain only mouse can be constructed by placing a murine germline IgH locus in a suitable vector as described in Example 1. The rearranged V_{H}DJ_{H} portion of the KT3 mAb is placed within the JH cluster and upstream of the murine μ constant region in the IgH locus, in place of the JDV segment shown in FIG. 5D. A first murine heavy chain constant region of the G1 subtype but truncated 5′ proximal to the codon coding for the cysteine present in the hinge region and involved in the interchain disulphide bridge, representing a sequence giving rise to a Fab portion and thus in turn also to produce F(ab′)2 antibodies, replaces the murine germline DNA that encodes the antibody heavy chain constant regions (IgG3, IgG1 and IgG2b shown in FIG. 5D) and is inserted immediately downstream of the murine germline DNA that represents Sγ3 switch sequence such that the human IgG1 region is operably linked to the murine Sγ3 switch sequence, and upstream of the Sε switch sequence. A second murine heavy chain γ constant region of the G1 subtype and also truncated 5′ proximal to the codon coding for the cysteine present in the hinge region and involved in the interchain disulphide bridge, representing a sequence giving rise to a Fab portion and thus in turn also to produce F(ab′)2 antibodies, and recombinantly joined to a linker and EGFP replaces the murine germline DNA that encodes the Cε antibody heavy chain constant region and is inserted immediately downstream of the murine germline DNA that represents Sε switch sequence such that the human Fab-encoding heavy chain constant region is operably linked to the murine Sε switch sequence, and upstream of the murine Sα switch sequence. The targeting construct is then placed into the germline locus of the mouse ES cell by homologous recombination to obtain a heavy-chain only animal, as in Example 1. Light chain animals are generated in a similar fashion, as in Example 1, for the KT3 antibody. Progeny animals obtained from a light-chain only animal and this heavy chain only animal will have B cells that produce an antibody having rearranged V_{H}DJ_{H} portion of the heavy chain from the KT3 antibody and (a) a truncated IgG constant region resulting in a Fab fragment when challenged with LPS, or (b) a truncated IgG constant region resulting in a Fab fragment and linked to a EGFP protein. Once the knockin mice are made, they may be immunized with a given antigen. In the process of deriving specific hybridomas, half of those cells growing well can be induced to switch to the “linker-EGFP” allowing the obtention at once of a green derivative of a given mAb.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

TABLE 1
Exemplary Humanized Antibodies

| Antibody | Citation |
|---|---|
| 0.5B | Maeda et al (1991) Hum. Antibod. Hybridomas 2: 124-134 |
| 1B4 | Singer et al (1993) J. Immunol. 150: 2844-2857 |
| 3a4D10 | Tempest et al (1994) Prot. Engng. 7: 1501-1507 |
| 425 | Kettleborough et al (1991) Prot. Engng. 4: 773-783 |
| 60.3 | Hsiao et al (1994) Prot. Engng. 7: 815-822 |
| A4.6.1 | Baca et al (1991) J. Biol. Chem. 272: 10678-10684 |
| AN100226m | Leger et al (1997) Hum. Antibod. 8: 3-16 |
| AT13/5 | Ellis et al (1995) J. Immunol. 155: 925-937 |
| AUK12-20 | Sato et al (1994) Mol. Immunol. 31: 371-381 |
| B1-8 | Jones et al (1986) Nature 321: 522-525 |
| B3{Fv}- | Benhar et al (1994) P.N.A.S. 91: 12051-12055 |
| PE38 | |
| B72.3 | Sha and Xiang (1994) Canc. Biother. 9: 341-349 |
| BMA 031 | Shearman et al (1991) J. Immunol. 147: 4366-4373 |
| BR96 | Rosok et al (1996) J. Biol. Chem. 271: 22611-22618 |
| BW431/26 | Gussow & Seemann (1991) Meth. Enzymol. 203: 99-121 |
| BrE-3 | Couto et al (1994)“Antigen and Antibody |
| | Molecular Engineering” pp: 55-59 |
| CC49 | Kashmiri et al (1995) Hybridoma 14: 461-473 |
| CTM01 | Baker et al (1994) “Antigen and Antibody |
| | Molecular Engineering” pp: 61-82 |
| Campath-1 | Riechmann et al (1988) Nature 332: 323-327 |
| Campath-9 | Gorman et al (1991) P.N.A.S. 88: 4181-4185 |
| D1.3 | Verhoeyen et al (1988) Science 239: 1534-1536 |
| D1.3 | Foote & Winter (1992) J. Mol. Biol. 224: 487-499 |
| DX48 | Lewis & Crowe (1991) Gene 101: 297-302 |
| Fd138-80 | Co et al (1991) P.N.A.S. 88: 2869-2873 |
| Fd79 | Co et al (1991) P.N.A.S. 88: 2869-2873 |
| H17E2 | Verhoeyen et al (1991) “Monoclonal Antibodies” |
| | pp: 37-43 |
| H52 | Eigenbrot et al (1994) Proteins 18: 49-62 |
| HCMV16 | Hamilton et al (1997) J. Infect. Diseases 176: 59-68 |
| HCMV37 | Tempest et al (1995) Int. J. Biol. Macromol. 17: 37-42 |
| HMFG1 | Verhoeyen et al (1993) Immunol. 78: 364-370 |
| JES1-39D10 | Cook et al, (1996) Prot. Engng. 9: 623-628 |
| K20 | Poul et al, (1995) Mol. Immunol. 32: 101-116 |
| M195 | Co et al (1992) J. Immunol. 148: 1149-1154 |
| M22 | Graziano et al (1995) J. Immmol. 155: 4996-5002 |
| MaE11 | Presta et al (1993) J. Immimol. 151: 2623-2632 |
| MikB1 | Hakimi et al (1993) J. Immmol. 151: 1075-1085 |
| N901 | Roguska et al (1996) Prot. Engng. 9: 895-904 |
| OKT3 | Adair et al (1994) Hum. Antibod. Hybridomas 5: 41-47 |
| PM-1 | Sato et al (1993) Canc. Res. 53: 851-856 |
| RSV19 | Tempest et al (1991) Biotech. 9: 266-271 |
| SK2 | Sato et al (1996) Hum. Antibod. Hybridomas 7: 175-183 |
| TES-C21 | Kolbinger et al (1993) Prot. Engng. 6: 971-980 |
| UCHT1 | Zhu and Carter (1995) J. Immunol. 155: 1903-1910 |
| YFC51.1 | Sims et al (1993) J. Immunol. 151: 2296-2308 |
| YTH12.5 | Routledge et al (1991) Eur. J. Immunol. 21: 2717-2725 |
| anti-B4 | Roguska et al (1996) Prot. Engng. 9: 895-904 |
| anti-Tac | Queen et al (1989) P.N.A.S. 86: 10029-10033 |
| mumAb4D5 | Carter et al (1992) P.N.A.S. 89: 4285-4289 |

TABLE 2
Hybridoma Fusion Partners

| | | |
|---|---|---|
| | | ATCC |
| Fusion Partners | Designation | Accession No. |

B Cell

| | | |
|---|---|---|
| Human B cells | A6 [A-6] | CRL-8192 |
| | F3B6 | HB-8785 |
| | GK-5 | CRL-1834 |
| | HuNS1 | CRL-8644 |
| | K6H6/B5 | CRL-1823 |
| | KR-12 | CRL-8658 |
| | LTR228 | HB-8502 |
| | MC/CAR | CRL-8083 |
| | MC/CAR-Z2 | CRL-8147 |
| | SHM-D33 | CRL-1668 |
| | SKO-007 | CRL-8033-1 |
| | SKO-007 [clone J3] | CRL-8033-2 |
| | WIL2-729HF2 | CRL-8062 |
| | WIL2-NS | CRL-8155 |
| | WIL2-S | CRL-8885 |
| Mouse B cells | 45.6.TG1.7 | CRL-1608 |
| | FO | CRL-1646 |
| | FOX-NY | CRL-1732 |
| | P3/NSI/1-Ag4-1 [NS-1] | TIB-18 |
| | P3X63Ag8 | TIB-9 |
| | P3X63Ag8.653 | CRL-1580 |
| | P3X63Ag8U.1 | CRL-1597 |
| | RPC5.4 | TIB-12 |
| | S194/5.XXO.BU.1 | TIB-20 |
| | Sp2/0-Ag14 | CRL-1581 |
| | Sp2/mIL-6 | CRL-2016 |
| Rat B cells | Y3-Ag 1.2.3 | CRL-1631 |
| | YB2/0 (YB2/3HL.P2.G11.16Ag.20) | CRL-1662 |

TABLE 3
Commonly used ligand/binding partner systems

| Binding partner | Ligand | Elutionₐ | Reference |
|---|---|---|---|
| Protein A | hIgG | Low pH | Nilsson et al. (1990) |
| | | | Methods Enzymol. |
| | | | 185: 144-161 |
| Z | hIgG | Low pH | Nilsson et al. (1987) |
| | | | Protein Eng. 1: 107- |
| | | | 113 |
| ABP | HSA | Low pH | Nygren et al. (1988) |
| | | | J. Mol. Recognit. |
| | | | 1: 69-74 |
| Hexahistidine | Bivalent metal | Imidazole/ | Porath et al. (1975) |
| 6 aa | chelator | low pH | Nature, 258: 598- |
| | | | 599 |
| GST | GSH | GSH | Smith et al. (1988) |
| | | | Gene 67: 31-40 |
| FLAG peptide | Monoclonal | EDTA/low pH | Hopp et al. (1988) |
| (8aa) | antibody M1 | | Bio/Technology |
| | | | 6: 1204-1210 |
| FLAG peptide | Monoclonal | Low pH | Brizzard et al. (1994) |
| (8aa) | antibody M2 | | BioTechniques |
| | | | 16: 730-735 |
| MBP | Amylose | maltose | di Guan et al. (1988) |
| | | | Gene 67: 21-30 |
| Pin Point_{b} | Streptavidin/ | biotin | Cronan, J. E. (1990) |
| | avidin | | J. Biol. Chem. |
| | | | 265: 10327-10333 |
| Bio_{c} (13 aa) | Streptavidin/ | Diaminobiotin | Schatz, P. J. (1993) |
| | avidin | | Bio/Technology 11, |
| | | | 1138-1143 |

Abbreviations : aa, amino acids; ABP, albumin-binding protein; GST, glutathione S-transferase; hIgG, human IgG; HSA, human serum albumin; mAb, monoclonal antibody; MBP, maltose-binding protein; Me2+, bivalent metal ion; FLAG, Asp-Tyr-Lys-Asp-Asp-Asp-Asp-Lys.
ₐMost common elution method.
_{b}Subunit of the transcarboxylase complex from Propionibacterium shermanii, biotinylated in vivo by E. coli.
_{c}Peptide selected from a combinatorial library and found to be biotinylated in vivo.

We claim:

1. A non-human animal having placed in its germline DNA at least: a rearranged variable region of a heavy chain of a human, non-human, chimeric or humanized lead antibody upstream of a native μ constant region and sequences encoding a heavy chain constant region that (i) replaces the native germline DNA that encodes one or more of the native heavy chain constant regions and (ii) is operably linked to a switch sequence, wherein the animal comprises a sequence arrangement as follows in its germline DNA:
5′-Sγ-human Cγ₁-S(ε or α)-human Cγ₂-3′
wherein S represents a switch sequence, Cγ₁ and Cγ₂ each represent a different human constant region γ subtype G1, G2, G3 or G4 or portion thereof, and each of Sε, Sα and Sγ are of murine origin.

2. The non-human animal of claim 1, wherein Sγ is Sγ3.

3. The non-human animal of claim 1, wherein Cγ₁ and Cγ₂ each represent a different human constant region γ subtype G1, G2, G3 or G4 or portion thereof.

4. The non-human animal of claim 1, wherein Cγ₁ and Cγ₂ each represent the same human constant region γ subtype G1, G2, G3 or G4 or portion thereof.

5. A non-human animal having placed in its germline DNA at least: a sequence arrangement encoding a heavy chain constant region that (i) replaces the native germline DNA that encodes one or more of the native heavy chain constant regions and (ii) is operably linked to a switch sequence, wherein the animal comprises a human γ heavy chain constant region sequence that replaces a murine γ heavy chain constant region and the animal comprises a sequence arrangement as follows in its germline DNA:
5′-Sγ-human Cγ₁-S(ε or α)-human Cγ₂-3′
wherein S represents a switch sequence, Cγ₁ and Cγ₂ each represent a human constant region γ subtype G1, G2, G3 or G4 or portion thereof, and each of Sε, Sα and Sγ are of murine origin.

6. The non-human animal of claim 5, wherein Sγ is Sγ3.

7. The non-human animal of claim 5, wherein Cγ₁ and Cγ₂ each represent a different human constant region γ subtype G1, G2, G3 or G4 or portion thereof.

8. The non-human animal of claim 5, wherein Cγ₁ and Cγ₂ each represent the same human constant region γ subtype G1, G2, G3 or G4 or portion thereof.

9. The non-human animal of claim 5, wherein the elements-Sα-Cα-are oriented 3′ of Cγ₂.

10. The non-human animal of claim 5, wherein Sα and Cα are of murine origin.

11. A non-human animal having placed in its germline DNA at least: a sequence arrangement encoding a heavy chain constant region that (i) replaces the native germline DNA that encodes one or more of the native heavy chain constant regions and (ii) is operably linked to a switch sequence, wherein the animal comprises a human γ heavy chain constant region sequence that replaces a murine γ heavy chain constant region and the animal comprises a sequence arrangement as follows in its germline DNA:
5′-Sμ-Cμ-Cδ-Sγ3-human Cγ₁-Sε-human Cγ₂-3′
wherein S represents a switch sequence, Cγ₁ and Cγ₂ each represent a human constant region γ subtype G1, G2, G3 or G4 or portion thereof, and each of Sε, Sα and Sγ are of murine origin.

12. The non-human animal of claim 11, wherein Cμ is of murine origin.
